SEQUENCE LISTING (3 sequences)

SEQ ID NO: 1 - protein, 31 aa, Artificial Sequence
  F3 peptide sequence
KDEPQRRSARLSAKPAPPKPEPKPKKAPAKK

SEQ ID NO: 2 - protein, 5 aa, Artificial Sequence
  CREKA peptide sequence
CREKA

SEQ ID NO: 3 - protein, 9 aa, Artificial Sequence
  Cyclic LyP-1 peptide
CGNKRTRGC

(19) United States
(12) Patent Application Publication
(10) Pub. No.: US 2010/0254914 A1
(43) Pub. Date: Oct. 7, 2010

(54) NANOWORMS FOR IN VIVO TUMOR TARGETING

(75) Inventors: Ji-Ho Park, La Jolla, CA (US); Lianglin Zhang, San Diego, CA (US); Austin M. Derfus, Solana Beach, CA (US); Michael J. Sailor, La Jolla, CA (US); Geoffrey A. Von Maltzahn, Somerville, MA (US); Todd Harris, Cambridge, MA (US); Sangeeta N. Bhatia, Lexington, MA (US); Dmitri Simberg, San Diego, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/712,552

(22) Filed: Feb. 25, 2010

Related U.S. Application Data
(60) Provisional application No. 61/155,415, filed on Feb. 25, 2009.

(51) Int. Cl. A61K 9/14; B32B 5/16; A61B 5/055; A61K 38/04; A61K 9/127; A61K 31/704; A61P 35/00; C01G 49/02; C12Q 1/02

(52) U.S. Cl. 424/9.322; 428/403; 514/1.2; 424/450; 514/34; 423/632; 435/29; 424/493; 977/700; 977/838; 977/811; 977/836; 977/892; 977/915; 977/927

(57) ABSTRACT

The disclosure provides elongated nanostructures useful for biological imaging and measurement. More particularly the disclosure provides nanoworms having an increased bioavailability compared to nanospheres.

### CROSS REFERENCE TO RELATED APPLICATIONS

This application claims priority under 35 U.S.C. §119 from Provisional Application Ser. No. 61/155,415, filed Feb. 25, 2009, the disclosure of which is incorporated herein by reference.

### STATEMENT AS TO FEDERALLY SPONSORED RESEARCH

The U.S. Government has certain rights in this invention pursuant to Grant Nos. CA119335, CA0124427, and N01-CO-37117 awarded by the National Institutes of Health.

### TECHNICAL FIELD

The disclosure provides elongated nanostructures useful for biological imaging, measurements, drug delivery and therapeutics. More particularly the disclosure provides nanoworms having an increased bioavailability compared to nanospheres.

### BACKGROUND

Tissue imaging, disease diagnostics and drug delivery are important for effective treatments.

### SUMMARY

The disclosure provides a composition comprising a plurality of nanostructure or particles conjugated or encapsulated to form an elongated structure having a first principle axis longer than the other two principle axes, with at least one dimension, such as length or diameter, between 1 and 200 nanometers (e.g., 1-100 nm, 1-50 nm, 5-50 nm etc.). In one embodiment, the nanoparticles are magnetic nanoparticles. In another embodiment, the nanostructure or particles comprise an iron oxide. The plurality of nanostructures can be encapsulated in a biocompatible material or conjugated to one another. In one embodiment, the biocompatible material is a dextran, polyethylene glycol, polyvinyl pyrrolidone or chitosan. In yet another embodiment, the nanostructure further comprises a targeting moiety linked to the nanostructure. For example, the targeting moiety can be a receptor ligand, an antibody, an antibody fragment or peptide comprising 2 or more amino acids.

The disclosure also provides a method of making an elongated nanostructure comprising precipitating a metal-containing nanoparticle in a high molecular weight dextran. In one embodiment, the method comprises precipitating iron oxide nanoparticles from a solution containing Fe²⁺_{(aq)}, Fe³⁺_{(aq)}, ammonia or other alkali solution (e.g., NaOH, KOH and the like), and a relatively low concentration of dextran.

The disclosure also provides methods for using a nanostructure of the disclosure for imaging a cell or tissue in vitro or in vivo. For example, the compositions and methods of the disclosure can be used for the imaging of cancer cells or tumors.

The disclosure provides worm-shaped dextran-coated iron oxide (magnetite or maghemite) nanoparticles. This disclosure provides materials comprising a chain-like aggregation of iron oxide (IO) cores (magnetic nanoworms; NW). The disclosure also demonstrates that such nanoworms can improve magnetic resonance contrast and in vivo tumor targeting properties over the well-known monocrystalline (spherical) dextran-coated IO nanoparticles. The chain-like aggregation of iron oxide cores also increases MRI sensitivity, demonstrating that NWs offer an improved ability to image very small tumors. The NW should be able to more efficiently deliver drugs to biological targets due to their large surface area, multiple attachment points, and long blood half-life.

The disclosure also provide methods for synthesis of worm-shaped dextran-coated iron oxide nanoparticles (nanoworms, NW) exhibiting substantial in vivo circulation times and significant tumor targeting when coated with tumor-homing peptides. Such worm-shaped nanoparticles home to tumors more efficiently than spherical both in vitro and in vivo. In one embodiment, the multivalent interactions between the targeting peptide-coated NW and their target molecules in tumor vasculature improves targeting compared to previous nanostructures. The surface chemistry, charge, and number of homing peptides are also found to be factors for improved homing and targeting. Additionally, NWs are found to display a greater magnetic resonance response than the spherical nanoparticles.

In one embodiment, the NW's of the disclosure comprise an increased surface area (compared to nanospheres) that can carry more homing peptides that more effectively interact with their tumor-based targets. The fact that the NW materials display a similar half-life in circulation compared to the smaller nanospheres (NS), even though they have comparable surface charge and chemistries is notable. The factors that lead to the highest circulation times and most effective targeting include: a neutral surface charge; complete and tight coverage of the iron oxide substructure with dextran and other hydrophilic polymers (e.g., PEG), and loading of targeting peptides. The chain-like aggregation of iron oxide cores also increases MRI sensitivity, suggesting that NW may offer an improved ability to image very small tumors. The NW should be able to more efficiently deliver drugs to biological targets due to their large surface area, multiple attachment points, and long blood half-life.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-D shows physical properties of magnetic iron oxide prepared as nanoworms (NW) compared with conventional spherical IO nanoparticle (Nanospheres; NS) preparations. (a) Transmission electron microscope image showing the worm-like nanostructure. More than 80% of the particles have a contorted linear appearance with a hydrodynamic length of 50-80 nm. Scale bar is 50 nm. Inset: atomic force microscope image showing the elongated shape. Scale bar is 100 nm. (b) Magnetization curves. The curves for NW, NS that resemble CLIO nanoparticles, and Feridex are shown. Feridex is a commercial preparation of IO nanoparticles that contains a dextran coating. (c) T₂ relaxation rates as a function of iron concentration (mM in Fe) for NW, NS and Feridex. (d) T₂-weighted MRI images of NW, NS and Feridex.

FIG. 2A-D shows internalization of magnetic nanoworms (NW) and nanospheres (NS) conjugated with an F3 tumor-homing peptide into MDA-MB-435 human breast cancer cells. (a) Hypothetical scheme showing the increase in multivalent interactions between receptors on the cell surface and targeting ligands on a NW and a NS. (b) Comparison of the efficiency of cellular internalization for various functionalized NW and NS systems. Internalization is quantified based on the fluorescence intensity from the Cy7-labeled particles. The approximate number of amine functionalities on each nanostructure is indicated next to the name abbreviation; e.g., “NW-42” corresponds to a magnetic nanoworm with 42 amine groups useable for peptide conjugation per worm, and “NS-7” corresponds to a magnetic nanosphere with 7 amine groups. The white bar denoted “NH₂” indicates the number of free amine groups attached to the dextran coating. The grey bar denoted “F3” indicates the number of F3 peptides conjugated to the amine groups, and the black bar denoted “PEG-F3” shows the number of peptides conjugated via a polyethylene glycol) linker (5 kDa) to the amine groups. The fluorescence signals were obtained in Cy7 channel (762 nm excitation/800 nm emission) using NIR fluorescence scanner (LI-COR biosciences) from microplate wells each containing about 10,000 cells, 2 hrs after incubation with the particles (40 μg total Fe in each well). Each nanostructure contains approximately the same number of Cy7 fluorophores per mole of iron atoms, conjugated to the dextran coating. All error bars show the standard deviation for three or more samples. (c) Fluorescence microscope images of cells 3 hrs after incubation with F3 (FITC)-conjugated NW (NW-175-F) or F3 (FITC)-conjugated NS (NS-30-F) (green). Nuclei were visualized with a DAPI stain (blue). Scale bar is 20 μm. (d) Three parameters were varied to determine optimal in vivo tumor targeting: the shape of the nanoparticle, the type of targeting ligand, and the nature of the molecular linker. Two types of surface linkers are used to attach targeting groups to iron oxide nanoworms (NW) (70) or spheres (60). A short hydrocarbon (30) places the targeting peptide (either F3 or CREKA, green lines in the image (50)) in close proximity to the dextran-coated nanostructure. A 5 kDa PEG linker places it further away. The number of targeting groups per nanoworm was varied to find the maximal in vivo circulation times and the optimal in vivo tumor targeting efficiency. These same chemistries were tested on iron oxide nanospheres; the NWs consist of several of these nanosphere cores linked together in a chain.

FIG. 3A-D shows in vivo behavior of non-targeted magnetic nanoworms (NW) in the mouse. (a) Percentage of NW remaining in circulation as a function of time, for two different injected doses (ID) of NW (mgFe/kg refers to the mass in mg of Fe in the dose relative to the body mass in kg of the animal) in nude Balb/c mice. The amount of Fe was quantified by SQUID measurement of the saturation magnetization in the blood extracted at each time point. (b) Biodistribution of NW 24 hrs after intravenous injection of 3 mgFe/kg into nude Balb/c mice. Bl, blood; Br, brain; H, heart; K, kidneys; Li, liver; Lu, lungs; LN, lymph node; Sk, skin; and Sp, spleen. (c) Fluorescence images (Bonsai fluorescence-imaging system, Siemens) showing in vivo biodistribution of Cy7-labeled aminated NW (NW-42) and NS (NS-7) in mice bearing MDA-MB-435 tumors. Arrows point to the tumors, and arrowheads point to the liver. Images were obtained in the Cy7 channel (762 nm excitation/800 nm emission) 6 hrs, 24 hrs, and 48 hrs after intravenous injection of the particles (1 mgFe/kg). (d) Plots comparing in vivo blood circulation half-life and tumor targeting efficiency of nanoworms as a function of CREKA targeting peptide density (mouse model). Blood half-life in mice without tumors is shown in the bottom plot, and percent of injected dose of nanoworms that target MDA-MB-435 and HT-1080 tumors are shown in the middle and the top plots, respectively. The effect of using a PEG linker to attach the CREKA targeting peptide (solid circles, “NW-P-C”) is compared with a short hydrocarbon linker (open circles, “NW-C”). Data obtained from SQUID measurements performed on blood or tissue samples, obtained 24 h post-injection. All error bars show the standard deviation for three or more animals.

FIG. 4A-D in vivo tumor targeting with CREKA-conjugated NW in mice bearing MDA-MB-435 or HT1080 tumors. (a) Blood half-life (left axis) and percentage targeted to MDA-MB-435 tumor (right axis) for CREKA-conjugated NW with and without a PEG linker in MDA-MB-435 tumors. (b) Blood half-life (left axis) and percentage targeted to tumor (right axis) as in (a), but using the HT1080 tumor model. The effectiveness of in vivo tumor targeting by NW is significantly related to the blood half-life and the number of conjugated CREKA peptides. The circulation time and tumor homing capability is best for the NW-P-175-C preparation (see Table 2, magnetic nanoworm containing a PEG linker, 175 amino groups, and 60 CREKA peptides). The targeted % ID/g of CREKA-conjugated NW was quantified by SQUID measurement of the magnetization values of the tumors removed 24 hrs after intravenous injection of the particles (3 mgFe/kg). The circulation data were acquired from the blood of nude Balb/c mice without tumors, injected with Cy7-labeled CREKA-conjugated NW. The Cy7 fluorescence intensity of blood extracted at each time point was determined with a NIR fluorescent scanner (LI-COR biosciences). (c) NIR fluorescence images of mice injected with Cy7-labeled CREKA-conjugated NW and control Cy7-labeled NW (NW-NH₂: NW-175, NW-PEG: NW-P175, NW-CREKA: NW-175-C, and NW-PEG-CREKA: NW-P175-C). The mice were imaged in the Cy7 channel (762 nm excitation/800 nm emission) 24 hrs after intravenous injection (ID: 1 mgFe/kg). Arrows point to the tumor, arrowheads point to the liver. (d) Histological images of Pegylated CREKA(FITC)-conjugated NW (NW-P175-C, green) in MDA-MB-435 and HT1080 tumors. CREKA-conjugated NW mainly colocalize with blood vessels (stained with CD31, red) in MDA-MB-435 tumors. In HT1080 tumors, the particles extravasate into tumor tissue (left panel) and also appear within blood vessels (right panel). Scale bar is 100 μm. All error bars show the standard deviation for three or more animals.

FIG. 5 shows physical and biological properties of large, highly branched NW prepared with high molecular weight dextran (MW 40,000, Sigma). Scale bar in the TEM image is 50 nm. These samples were not tested for their tumor-homing properties in the present study.

FIG. 6 shows transmission Electron Microscope (TEM) images showing the shape and size of NS and Micromod samples. The shape and size of the NS made in this work are similar to iron oxide nanoparticles with a cross linked dextran coating reported previously (CLIO). They display a spherical morphology with a relatively narrow size distribution)-4. Micromod samples appear as clusters of IO cores, rather than the chain-like structures seen with NW. Scale bar is 50 nm.

FIG. 7A-B shows quantification of fluorescence. (a) Quantification of fluorescence intensity in MDA-MB-435 cells incubated with Cy7-labeled F3-conjugated NW or NS for the indicated times. The fluorescence signals were obtained in Cy7 channel (762 nm excitation/800 nm emission) using NIR fluorescence scanner (LI-COR biosciences) from microplate wells each seeding about 10,000 cells overnight, after incubation with the particles (40 μg total Fe in each well). (b) Comparison of fluorescence intensity and magnetization with the cells internalized with F3-conjugated NW or NS after 2 hrs of incubation. Magnetization was measured by SQUID on freeze-dried cells. The data are normalized to the saturation magnetization value of an equal quantity of cells treated with a blank PBS solution. All error bars show the standard deviation for three or more samples.

FIG. 8 shows passive accumulation of NW or NS in tumors of mice bearing the MDA-MB-435 or HT1080 tumors, quantified by SQUID. The tumors were harvested 24 hrs after intravenous injection of the particles (3 mg Fe/kg body mass), freeze-dried, and then analyzed using the SQUID magnetometer. All error bars show the standard deviation for three or more animals.

FIG. 9A-B show in vivo data. (a) In vivo circulation of F3-conjugated NW of different formulations 30 min after intravenous injection into nude Balb/c mice. All F3-conjugated NW tested are cleared from the blood stream in <1 hour. (b) NIR fluorescence images of the mice bearing MDA-MB-435 and HT1080 tumors, injected with Cy7-labeled NW conjugated with F3 or CREKA. The mice were imaged 24 hrs after an intravenous injection of the particles (1 mg Fe/kg body mass) using a Bonsai fluorescence-imaging system. Arrows point to the tumors, and arrowheads point to the liver. No tumor homing is detectable with the F3 targeting peptide, whereas the tumors are clearly visualized with the CREKA formulations. All error bars show the standard deviation for three or more animals.

FIG. 10A-E show in vivo measurements and biodistribution data. (a) Quantification of in vivo tumor targeting capabilities of unmodified and CREKA-conjugated NW, NS and Micromod particles in mice bearing MDA-MB-435 tumors. The targeting efficiency (% ID/g) was analyzed by SQUID measurement of saturation magnetization values of the tumors 24 hrs after particle injection (3 mg Fe/kg body mass). (b) Representative biodistribution of Cy7-labeled CREKA-conjugated NW and NS in MDA-MB-435 tumor mice. The images were acquired by NIR fluorescence imaging of the organs harvested 24 hrs after intravenous injection of the particles (1 mg Fe/kg body mass). Br, H, K, Li, Lu, T, and Sp represent brain, heart, kidney, liver, lung, tumor and spleen, respectively. The tumors were cut in half for the imaging. Note that the NW-P175-C formulation (with the highest targeting efficiency measured in this study) exhibits tumor homing regardless of the size of the tumor (examples of 0.2 cm, 0.5 cm, and 1 cm tumors are shown). (c) Fluorescence images showing colocalization of CREKA (FITC)-conjugated NW (NW-P175-C, green) and anti-fibrin(ogen) stain (red) in the blood vessels and stroma of MDAMB-435 and HT1080 tumors. Scale bar is 20 μm. (d) SQUID quantification of biodistribution of unmodified NW, PEGylated NW-C (NW-P175-C), and NW-C (NW-175-C) in mice bearing MDA-MB-435 tumors, obtained 24 h post-injection. (e) SQUID quantification of in vivo tumor targeting efficiency of NW, NS and Micromod samples with and without CREKA targeting peptide, in mice bearing MDA-MB-435 tumors, 24 h post-injection. All error bars show the standard deviation for three or more animals.

FIG. 11 shows a comparison of targeting efficiency of PEGylated CREKA-conjugated NW (NW-P-C) and PEGylated KAREC (scrambled version of CREKA)-conjugated NW (NW-P-K) in MDA-MB-435 tumor-bearing mice as a function of peptide number per NW, 24 h post injection. Note that targeting efficiency of NW-P-C with ˜60 CREKA peptides is significantly greater than that of NW-P-K with ˜60 KAREC peptides (p<0.05).

FIG. 12A-D shows characterization of the components of cooperative nanosystems. (a) Schematic showing the components of the two cooperative nanomaterials systems used in this study. The first component consists of gold nanorods (NR), which act as a photothermal sensitizer. The second component consists of either magnetic nanoworms (NW), or doxorubicin-loaded liposomes (LP). Irradiation of the NR with a NIR laser induces localized heating that stimulates changes in the tumor environments. The NW or LP components decorated with LyP-1 tumor targeting peptides bind to the heat-modified tumor environments more efficiently than to the normal tumor environments. Transmission electron microscope images of all three components are shown. Scale bars indicate 50 nm. (b) Temperature changes induced by localized laser irradiation (+L) of mice injected with NR alone (no NW or LP). Tumor-bearing mice were injected intravenously with either PEGylated NRs (NR) or saline (saline). Trace labeled “NR-L” is a control where NRs were injected but the tumor was not irradiated. Data and images obtained 72 h post-injection; infrared thermographic maps of average tumor surface temperature were obtained after laser exposure for the indicated times. Scale bar indicates 1 cm. (c) Effect of heating time on p32 expression in MDA-MB-435 xenograft tumor. Tumor in an athymic (nu/nu) mouse was heated at 45° C. for 30 min in a water bath. Images at left show cell surface p32 immunostaining of tumor sections 6 hrs post-treatment. Symbols + and − indicate with and without heating, respectively. Scale bar indicates 50 μm. At right are western blot results for p32 relative to β-actin control. * indicates P<0.05 for 0 h and 6 h intensity ratio (n=3˜4). (d) Fluorescence microscope images of C8161 or MDA-MB-435 cells probing in vitro cellular binding and internalization of Lyp1-conjugated Cy5.5-labeled magnetic nanoworms (Lyp1NWs, in green) upon heating to 45° C. Samples were incubated for 20 min at 37° C. (−) or 45° C. (+) and then held at 37° C. for an additional 2 hrs. Cell nuclei and p32 stained with 4′-6-diamidino-2-phenylindole (DAPI, blue), and anti-p32 antibody followed by Alexa Fluor® 594 goat anti-rabbit IgG antibody (red), respectively. Scale bar indicates 50 μm. All error bars indicate standard deviations from >3 measurements.

FIG. 13 shows the effect of p32 expression in C8161 xenograft tumor on increased temperature and heating time in vivo. A C8161 xenograft tumor on athymic (nu/nu) mouse was heated at 45° C. for 30 min using temperature-controlled water bath. At 6 h after heating treatment, the tumor sections were imaged for analysis of p32 expression by immunostaining (Left). At different time period after heating treatment, the tumors were harvested and processed for analysis of p32 expression by western blot (Right). β-actin was used as a control. Symbols − and + indicate no heating (37° C.) and heating (45° C.), respectively. (n=3).

FIG. 14A-B shows the effect of p32 expression in various cultured cells on increased temperature. (A) Immunoblots of p32 expression on various cultured cells at increased temperature. C8161, HeLa, MDA-MB-435, and MDA-MB-231 cells were treated for 20 min at 37° C. or 45° C. (in cell incubator) and then incubated for an additional 2 h at 37° C. β-actin was used as a control. (B) Fluorescence images of p32 expression on the surface of various cultured cells at increased temperature by immunostaining. The experimental procedure was the same as in (A). For immunostaining, after washing cells with PBS three times, the cells were fixed with 4% paraformaldehyde for 20 min, and blocked with the solution containing 1% BSA in PBS for 30 min, incubated with 5 μg/mL anti-p32 antibody for 1 h, and then with 5 μg/mL Alexa Fluor® 594 goat anti-rabbit IgG antibody for 1 h at room temperature. The nuclei stained with DAPI were observed in blue channel (excitation at 360 nm/emission at 460 nm). The p32 were observed in Cy3.5 channel (excitation at 580 nm/emission at 620 nm). Symbols − and + indicate no heating (37° C.) and heating (45° C.), respectively.

FIG. 15A-C shows temperature-induced amplification of in vivo tumor targeting. (a) Fluorescence intensity from Cy7-labeled LyP1-conjugated magnetic nanoworms (LyP1NW) and Cy7-labeled control nanoworms (NW) in MDA-MB-435 tumor as a function of externally applied heat (30 min). Heated at (45° C.) and unheated (37° C.) samples indicated with (+) and (−), respectively. The tissues were collected from the mice 24 hrs post-injection; NIR fluorescence images use Cy7 channel. * indicates P<0.05 (n=3-4). (b) Fluorescence image of major organs from the mice in (a). T+, T−, Li, Sp, K, and Br indicate heated tumor, unheated tumor, liver, spleen, kidney, and brain, respectively. (c) Histological analysis of LyP1NW or NW distribution in MDA-MB-435 tumors with (+) or without (−) application of external heat. Green indicates NWs (labeled with Cy 5.5). Cellular stains same as in FIG. 1d, blood vessels stained with CD31 followed by Alexa Fluor® 594 goat anti-rat IgG. Arrowhead indicates a lymphatic vessel structure displaying a signal from the labeled LyP1NWs. Scale bar is 100 μm. Error bars indicate standard deviations from >3 measurements.

FIG. 16A-B shows heat-mediated cytotoxicity of targeted therapeutic nanoparticles in vitro. (a and b) Temperature-induced cytotoxicity of various therapeutic molecule or nanoparticle formulations toward MDA-MB-435 human carcinoma cells by MTT assay. The cells were treated with free DOX, control DOX-containing liposomes (LP), or LyP1-conjugated, DOX-containing liposomes (LyP1LP) with the indicated concentrations of DOX. Samples incubated at 37° C. (a) or 45° C. (b).

FIG. 17A-D shows successful anti-tumor therapy using cooperative nanosystem, demonstrated in mice bearing MDA-MB-435 tumors. (a) Quantification of in vivo accumulation of DOX in tumors as a function of NR-mediated laser heating of Lyp1-conjugated liposomes (LyP1LP) or control liposomes that contain no targeting peptide (LP). NR+L and NR−L indicate mice containing gold nanorods that were or were not subjected to laser treatment, respectively. Amount of DOX present quantified by fluorescence microscopy to yield a percentage of injected dose per tissue mass. * indicates P<0.05 (n=3˜4). (b) Histological analysis of DOX distribution in tumors from the mice in (a) who were subjected to NR-mediated thermal therapy showing the distribution of nanoparticles (Alexa Fluor® 488 label on control liposome and 5(6)-carboxyfluorescein (FAM) label on LyP1, green) and DOX (red). Nuclei stained with DAPI (blue). Scale bar is 100 μm. (c) Change in tumor volume of different treatment groups containing bilateral MDA-MB-435 xenograft tumors. 72 hrs post-injection of gold nanorods (NR, 10 mg Au/kg), mice were injected with a single dose of saline, control liposomes (LP), and LyP1-conjugated liposomes (LyP1LP). “+H (Hyperthermia)” denotes one of the two tumors in the animal that was irradiated with the NIR laser. The tumor not irradiated is indicated as “−H”. Tumor volumes monitored every 3 days post-irradiation. Error bars indicate standard deviations from >3 measurements. * indicates P<0.05 and ** indicates P<0.02 for +H+LyP1LP sample and all other treatment sets (n=4˜6). (d) Survival rate in different treatment groups after a single dose (3 mg DOX/kg) into mice (n=6) containing single MDA-MB-435 xenograft tumors. Error bars indicate standard deviations from >3 measurements.

### DETAILED DESCRIPTION

As used herein and in the appended claims, the singular forms “a,” “and,” and “the” include plural referents unless the context clearly dictates otherwise. Thus, for example, reference to “a nanostructure” may include a plurality of such nanostructures and reference to “the nanoworm” may include reference to one or more nanoworms, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

Tumorigenesis is a multi-step process that requires expression of tumor-associated proteins and suppression of proteins controlling normal cell growth. Many of the identified tumor-specific proteins have been exploited to develop powerful antibody, aptamer, peptide, and small molecule-based ligands for targeting of diagnostic or therapeutic agents. Ultrasensitive in vivo imaging for early detection of cancers and efficient delivery of therapeutics to malignant tumors are two primary goals in cancer bionanotechnology. However, the development of non-toxic, functional nanoparticles that can successfully home to tumors presents some significant challenges. Dextran-coated magnetic iron oxide (IO) nanoparticles are of particular interest because they show relatively low toxicity and long in vivo circulation (˜10 hrs) and they dramatically enhance hydrogen T2 relaxation in magnetic resonance imaging (MRI). The clinical power of these materials may be amplified by improving MRI relaxivity, blood circulation times, and the homing of such nanoparticles to tumors.

Ligand-directed targeting of therapeutic nanomaterials has been widely pursued to improve therapeutic efficacy, although limitations imposed by the tumor microenvironment, such as restricted transvascular transport and receptor accessibility and clearance of targeted nanoparticles comprising such antibody, aptamer, peptide and small molecule have prevented realization of their full capabilities. Although the porous microstructure of tumor blood vessels is favorable for non-specific infiltration of circulating nanomaterials into the extravascular region of the tumor, extravasated nanomaterials are generally deposited close to the vessels, resulting in a highly heterogeneous distribution of therapeutic agents in the tumor.

Hyperthermia has been reported to not only improve nanoparticle extravasation in tumors, but it also can selectively damage neoplastic cells to activate immunological processes and induce expression of particular proteins. Use in the clinical setting in concert with chemotherapy and radiotherapy, tumor-specific hyperthermia would be a powerful tool to manipulate tumor microenvironments in order to enhance the interactions between cancerous tissues and therapeutic agents. However, hyperthermia methods in clinical practice lack intrinsic specificity for tumor tissues, requiring complex implementation strategies and frequently resulting in exposure of large volumes of normal tissues to hyperthermic temperatures alongside tumors. Gold nanorods (NRs), for example, passively accumulated in tumors via their fenestrated blood vessels. The accumulated NRs can be used to precisely heat tumor tissues by amplifying their absorption of otherwise benign near-infrared energy and allow the recruitment and more effective penetration of a second, specifically targeted nanoparticle. As discussed more fully below, the disclosure provides not only a single therapy comprising a nanoworm, but also a cooperative nanomaterials system, wherein NWs accumulated in a tumor photothermally activate the local microenvironment to amplify the targeting efficacy of two types of targeted, circulating nanoparticles: magnetic nanoworms (NWs) and liposomes (LPs) loaded with the anti-cancer drug doxorubicin (DOX). Other liposomal or micellar formulation may be used with any number of different chemotherapeutic agent. For example, the chemotherapeutic agent can be doxorubicin, taxol, combretastatin or any combination thereof.

Efforts to increase MRI sensitivity have focused on development of new magnetic core materials or improvements in nanoparticle size or clustering. However, most efforts to improve the morphological characteristics of such nanoparticles have resulted in materials with relatively short blood half-life (1˜2 hrs) due to incomplete additional hydrophilic coatings. While decoy particles that bind to plasma opsonins can be used to improve the circulation time of nanoparticles by blocking uptake by the mononuclear phagocytic system (MPS), it is more desirable to incorporate an inherent ability to avoid the MPS in the nanoparticle itself.

At the micro scale, particle shape plays a dominant role in particle uptake by phagocytes. A study on the uptake of gold nanoparticles into cultured tumor cells concluded that spherically shaped particles have a higher probability of cell internalization than rod-shaped particles. When nanoparticles are used in vivo, one of the most important issues is to avoid clearance by the MPS, which is primarily located in the liver.

The disclosure demonstrates that nanoparticles with elongated shapes exhibit unique in vivo behavior such as low liver uptake and, as a result, prolonged blood half-life. The disclosure demonstrates that a nanostructure with an elongated assembly of metallic cores such as iron oxide (IO) cores provides long in vivo circulation times and that this improves homing of the particles to tumors. High aspect ratio nanomaterials such as carbon nanotubes and worm micelles have been found to circulate in vivo long enough to enable homing to biological targets despite their micron-sized length. In addition, pseudo one-dimensional assemblies of nanocrystals can display desirable optical or magnetic properties not found in the isodimensional materials. The disclosure demonstrates that a chain-like aggregation of metallic cores such as iron oxide (IO) cores (magnetic nanoworms; NW) can improve magnetic resonance contrast and in vivo tumor targeting properties over the well-known monocrystalline dextran-coated IO nanoparticles. The improved targeting characteristics can be attributed, in part, to an increased surface area that can carry more homing peptides that more effectively interact with their tumor-based targets. The fact that the NW materials display a similar half-life in circulation compared to the smaller nanospheres (NS), even though they have comparable surface charge and chemistries is notable. Some of the factors that lead to the highest circulation times and most effective targeting include, but are not limited to: a neutral surface charge; complete and tight coverage of the iron oxide substructure with dextran and other hydrophilic polymers (e.g., PEG), and an optimal loading of targeting peptides. The data presented here indicates that these factors are important for long blood half-life. The chain-like aggregation of the magnetic cores (e.g., the iron oxide cores) also increases MRI sensitivity, suggesting that NW may offer an improved ability to image very small tumors. Several methods to construct one-dimensional assemblies of nanocrystals are known in the art, for example, methods that involve the use of molecular coatings or biotemplates. These approaches appear to provide means to control the chain-like nanostructures fairly precisely.

The disclosure demonstrates that tailoring the shape, as well as the size and charge, can improve in vivo tumor targeting capability of a nanomaterial. This elongated assembly of metals such as iron oxide cores coated by a polymeric material such as dextran is analogous to certain strand-like viruses or biomolecules that display long residence times in the blood stream. The NW should be able to more efficiently deliver drugs to biological targets due to their large surface area, multiple attachment points, and long blood half-life.

The disclosure provides conjugated beads or elongated metallic nanostructures (generally referred to herein as “elongated nanostructure”. As used herein, the term “elongated nanostructure” refers to various materials having one principle axis longer than the other two principle axes, such as a cylindrical or tubular configuration, with at least one dimension, such as length or diameter, between 1 and 100 nanometers. Such elongated nanostructures are capable of MRI detection and imaging as well as photothermal activation. The metallic nanostructure can comprise any metal or alloys thereof.

Metals, alloys and materials useful for the formation of a nanostructure of the disclosure can be obtained based upon a functional layer or thermal bias layer. The material can be selected from the group of noble metal and transition metal including, but not limited to, Ag, Au, Cu, Al, Fe, Co, Ni, Ru, Rh, Pd, and Pt. In another embodiment, the material comprises Fe. A further surface functional layer can be added or formed in combination with the noble or transition metal core material. Such functional layers can include, but are not limited to, Ag oxide, Au oxide, SiO₂, Al₂O₃, Si₃N₄, Ta₂O₅, TiO₂, ZnO, ZrO₂, HfO₂, Y₂O₃, Tin oxide, antimony oxide, and other oxides; Ag doped with chlorine or chloride, Au doped chlorine or chloride, Ethylene and Chlorotrifluoroethylene (ECTFE), Poly(ethylene-co-butyl acrylate-co-carbon monoxide) (PEBA), Poly(allylamine hydrochloride) (PAH), Polystyrene sulfonate (PSS), Polytetrafluoroethylene (PTFE), Polyvinyl alcohol (PVA), Polyvinyl chloride (PVC), Polyvinyldene fluoride (PVDF), Polyvinylprorolidone (PVP), and other polymers; stacked multiple layers at least two layers including above listed metal layers and non-metal layers, and the like. A typical material is a metal such as Au, Ag, Fe, Ti, Ni, Cr, Pt, Ru, Ni—Cr alloy, NiCrN, Pt—Rh alloy, Cu—Au—Co alloy, Ir—Rh alloy or/and W—Re alloy. The material used should be biocompatible.

The geometry or structure of the nanomaterial can incorporate the functional capabilities of nanotip, nanosphere, and nanoring geometries. Other geometries can include spherical, circular, triangle, quasi-triangle, square, rectangular, hexagonal, oval, elliptical, rectangular with semi-circles or triangles and the like. However, the structure(s) should be linked or have an elongated structure. The nanostructures of the materials and geometries ideally have an absorbance or excitation wavelength in the near infrared range. Selection of suitable materials and geometries are known in the art. Excitation at longer wavelengths provides deeper penetration into tissue with minimal photothermal damage.

Various nanostructure geometries are capable of near-infrared (NIR) excitation. For example, crescents, bowls, hollow spheres and the like have a higher local field-enhancement factor in the near-infrared wavelength region due to the simultaneous incorporation of SERS hot spots, leading to the strong hybrid resonance modes from nanocavity resonance modes.

One of skill in the art will recognize that the size, shape, and thickness or where multi-layers are present layer thickness can all be individually controlled by modifying the size of a sacrificial nanostructure template, the deposition angle, the deposited layer thickness, and the material of each layer. In one embodiment, the nanostructure comprises a spherical or semi-spherical structure commonly produced in the art.

The metallic composition of composite nanostructures of the disclosure are biocompatible, and thus can be bio-functionalized.

The term “functionalized” is meant to include structures with one, two or more layers of different metals, structures with functional groups attached thereto, and the like. For example, to form a linkage to a peptide, oligonucleotide or other biomaterial, to prolong or target analyte interaction with a noble metal nanostructure, a binding agent/targeting domain can be used to promote interaction of a nanostructure with a desired target. An alkanethiol, such as 1-decanethiol, can be used to form the capture layer on the noble metal (Blanco Gomis et al., J. Anal. Chim. Acta 436:173 [2001]; Yang et al., Anal. Chem. 34:1326 [1995]). Other exemplary capture molecules include longer-chained alkanethiols, cyclohexyl mercaptan, glucosamine, boronic acid and mercapto carboxylic acids (e.g., 11-mercaptoundecanoic acid).

In one embodiment, the elongated or conjugated beads are encapsulated or linked to dextran or other polymeric materials that are useful for increasing the circulating half-life or stability in vivo. Other polymeric materials can be selected from the group, but are not limited to, polyethylene glycol (PEG), a lipid, chitosan, zein, polylactic acid, polyglycolic acid, collagen, fibrin, co-polymers of polylactic acid and polyglycolic acid, and co-polymers of dextran and polylactic acid. In a further embodiment, the elongated nanostructure is linked to a targeting moiety or plurality of targeting moieties (e.g., a peptide ligand, antibody, antibody domain, receptor, receptor fragment and the like) to target the nanostructure to a particular cell type or tissue. In some embodiments, the ligand is a peptide. In another embodiment, the peptide has a density that maintains free amine groups at a minimum and maintains the coverage of the underlying nanoparticle. In a further embodiment, the peptide is an F3 peptide or a CREKA (SEQ ID NO:2) peptide. In yet another embodiment, the CREKA peptide comprises less than about 60 peptides per elongated nanostructure. Table 1, for example, depicts certain elongated nanodevice characteristics:

TABLE 1
Characteristics of targeted and untargeted NWs and NSs.

Number of peptides

| | Size | Targeting | per NW | per g Fe |
|---|---|---|---|---|
| Sample^{[a]} | [nm]^{[b]} | peptide | or NS^{[c]} | (×10²⁰)^{[d]} |
| NS | 30.3 | none | | |
| NS-30-C | 34.3 | CREKA | 18 | 9.4 |
| NS-P30-C | 46.8 | CREKA | 13 | 6.8 |
| MM-500-C | 107.2 | CREKA | 350 | |
| NW | 68.7 | none | | |
| NW-42-F | 73.7 | F3 | 23 | 1.7 |
| NW-P42-F | 87.3 | F3 | 16 | 1.2 |
| NW-175-F | 76.6 | F3 | 69 | 5.1 |
| NW-P175-F | 88.2 | F3 | 48 | 3.0 |
| NW-350-F | 76.1 | F3 | 83 | 6.2 |
| NW-P350-F | 90.8 | F3 | 59 | 4.4 |
| NW-42-C | 70.9 | CREKA | 29 | 1.6 |
| NW-P42-C | 82.4 | CREKA | 23 | 1.2 |
| NW-175-C | 70.2 | CREKA | 117 | 6.3 |
| NW-P175-C | 85.0 | CREKA | 60 | 3.2 |
| NW-350-C | 72.3 | CREKA | 205 | 10.2 |
| NW-P350-C | 85.5 | CREKA | 90 | 4.9 |

^{[a]}The number following the letter identifier designates the number of amine groups per particle. The letter P indicates that a PEG spacer is used. The -F or -C suffix denotes an F3- or CREKA-conjugated particle, respectively. For example, NW-P175-C denotes a NW with 175 amines to which CREKA is conjugated through a PEG spacer. MM = aminated Micromod. Micromod is a commercially available IO nanoparticle preparation.
^{[b]}Mean hydrodynamic size based on dynamic light scattering measurements.
^{[c]}Number of targeting peptides per single NW or NS.
^{[d]}Number of targeting peptides (×10²⁰) per gram of Fe.

Other conjugate moieties include proteins, peptides, and peptide mimetics. In one aspect, members from this group of moieties are selected based on their binding specificity to a ligand expressed in or on a target cell type or a target organ. Alternatively, moieties of this type include a receptor for a ligand on a target cell (instead of the ligand itself), and in still other aspects, both a receptor and its ligand are contemplated in those instances wherein a target cell expresses both the receptor and the ligand. In other embodiments, members from this group are selected based on their biological activity, including for example enzymatic activity, agonist properties, antagonist properties, multimerization capacity (including homo-multimers and hetero-multimers). With regard to proteins, conjugate moieties contemplated include full length protein and fragments thereof which retain the desired property of the full length proteins. Fusion proteins, including fusion proteins wherein one fusion component is a fragment or a mimetic, are also contemplated. This group also includes antibodies along with fragments and derivatives thereof, including but not limited to Fab′ fragments, F(ab)₂ fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

Cell receptor ligands useful for targeting include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g., inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, glucagon, G-protein coupled receptor ligands, etc.

Accordingly any number of targeting ligands can be conjugated to the nanostructure (e.g., a receptor bound to the surface of a nanostructure that interacts reversibly or irreversibly with a specific analyte). Alternatively or in addition an uptake moiety can be linked to the nanoparticle (e.g., a TAT moiety, see, for example, International Patent Publ. No. WO/2007/095152). Examples of targeting ligands include antigen-antibody pairs, receptor-ligand pairs, and carbohydrates and their binding partners. Binding ligands to a wide variety of analytes are known or can be readily identified using known techniques. As will be appreciated by those in the art, any two molecules that will associate, may be used, either as the analyte or the functional group (e.g., targeting/binding ligand). Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. In one embodiment, the binding ligands are portions (e.g., the extracellular portions) of cell surface receptors.

The disclosure thus provides elongated nanostructures comprising a plurality of individual nanostructures linked to one another. Typically the nanostructure comprise a metallic nanosphere. The nanosphere may comprise any number of different metals or allows such as, but not limited to, gold, silver, copper, iron and alloys and combinations thereof. The nanostructure may be further coated in a polymeric material that improves circulatory time in vivo. The elongated nanostructure my further comprise a targeting ligand or plurality of targeting ligands. The targeting ligands may be identical or different. Furthermore, In one embodiment, the disclosure provides synthesis and biological application of worm-shaped dextran-coated nanoparticles (nanoworms, NW) exhibiting prolonged in vivo circulation times and improved tumor targeting when coated with tumor-homing peptides compared to spherical nanostructures. The synthesis of NW was based on the observation that nanostructures such as magnetic nanoparticles can become aligned along strands of high molecular weight dextran, and the nanoparticle geometry (worm-shaped vs spherical) affects their efficacy both in vitro and in vivo. This can be attributed to the improvement in tumor homing to prolonged in vivo circulation and enhanced multivalent interactions between the targeting peptide-coated NW and their target molecules in tumor vasculature.

In one embodiment, nanoparticles comprising iron oxide are produced from a mixture of iron (II) chloride and iron (III) chloride with a polysaccharide (e.g., Ficoll™) in water, by treatment with base (e.g., NaOH or NH₄OH) and heating under an inert atmosphere.

Furthermore, the disclosure demonstrates that for a constant ratio of attached targeting peptides per iron atom, NW display a greater ability to be taken up by cultured tumor cells than NS. These results suggest that NW will also facilitate the in vivo homing of multivalent ligands to biological targets. Two peptides (F3 and CREKA) were chosen for in vivo tumor targeting study, because they are known to recognize different tumor targets. F3-conjugated NW exhibited rapid MPS clearance regardless of the protecting or attachment chemistries used. This rapid clearance is attributed to the large number of positively charged residues on the relatively large F3 peptide. Other peptides that can be used for targeting including antibodies, antibody fragments, receptor proteins and fragments, ligand binding proteins and moieties (e.g., including soluble polypeptide/peptide domains derived from transmembrane proteins) and the like.

The short peptide CREKA endows superior targeting capability to the NW. PEGylated NW conjugated with the appropriate number of CREKA targeting moieties circulate in vivo for a long period (blood half-life of over 12 hrs), and prominent tumor uptake is observed in both MDA-MB-435 and HT1080 tumors. Other targeting-molecules can be used in place of or in addition to CREKA, however, it is suggested that the blood half-life of the targeting molecule-nanomaterial ensemble must be considered when selecting the appropriate ligand for in vivo tumor targeting when several ligand candidates with similar targeting affinity are available. CREKA, a short linear peptide which is likely non-immunogenic and is neutrally charged, maintains its binding to blood clots (fibrin(ogen)) when coupled to PEGylated NW. Furthermore, it displays the same self-amplifying homing behavior seen previously with CREKA-conjugated IO nanoparticles.

The methods of the disclosure are useful for treating diseases or disorders comprising cell proliferative diseases or disorders, inflammation, tissue damage and the like. For example, the methods and compositions of the disclosure are useful for treating or studying cell proliferative disorder such as cancer, inflammatory disorder and autoimmune disorders to name a few.

The nanoworms and elongated nanostructures of the disclosure can also be used in a combination therapy comprising hyperthermia and drug delivery. For example, as described herein, the elongated nanostructures (including nanoworms) have improved circulatory times, and reduced clearance. In addition, the structures can be effectively targeted to tumors and other tissues by conjugating the elongated nanostructure to a targeting ligand (e.g., a peptide etc.). The elongated nanostructures can then be localized by magnetic forces (where the nanostructure comprises a magnetic metal) and/or through ligand binding at a desired site. The nanostructures can then be excited to thermally modify the tissue, increasing vasculature and damaging the desired tissue. Following and simultaneously with the delivery of the elongated nanostructure and targeted chemotherapeutic agent (e.g., a chemotherapeutic small molecule, antibody, peptide or the like) can be administered to the subject. The chemotherapeutic, for example, may be formulated in a targeted liposome. In one embodiment, the disclosure demonstrates the doxorubicin liposomes can be used. The liposomes may further comprise a targeting ligand (e.g., the same targeting ligand used on the elongated nanostructures) to cause the targeted delivery of the liposome's payload to the desired tissue.

Accordingly, the disclosure provides a method for treating a cell proliferative disorder comprising a tumor by administering a targeted nanoworm to the subject, thermally treating the tumor site by thermally activating the nanoworm and contacting the subject with a chemotherapeutic agent. In one embodiment, the chemotherapeutic agent comprises a targeted liposome containing the chemotherapeutic agent.

A nanostructure comprising a NW or elongated structure can be formulated in pharmaceutically acceptable carrier. Pharmaceutically acceptable carriers useful for administration to a cell, tissue or subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

In some embodiments, the disclosure provides kits and systems for tissue imaging and drug delivery.

Excitation of the nanostructures of the disclosure can be performed by contacting the nanostructure with appropriate electromagnetic radiation (e.g., an excitation wavelength). Wavelengths in the visible spectrum comprise light radiation that contains wavelengths from approximately 360 ran to approximately 800 run. Ultraviolet radiation comprises wavelengths less than that of visible light, but greater than that of X-rays, and the term “infrared spectrum” refers to radiation with wavelengths of greater 800 nm. Typically, the desired wavelength can be provided through standard laser and electromagnetic radiation techniques.

The following examples are intended to illustrate but not limit the disclosure. While they are typical of those that might be used, other procedures known to those skilled in the art may alternatively be used.

### EXAMPLES

### Example 1

Preparation and physical characterization of magnetic nanoworms. The synthesis of magnetic nanoworms (NW) involves precipitation of IO nanoparticles from a solution containing Fe²⁺_{(aq)}, Fe³⁺_{(aq)}, ammonia or other alkali solution (e.g., NaOH, KOH and the like), and a relatively low concentration of dextran. The dextran used was of a greater molecular mass than typically employed in such preparations (20 kDa vs. 10 kDa). Suitable dextran for use can be from 10 to 30 kDa. Nanoworms (NW) were synthesized using a modification of the published preparation of dextran-coated iron oxide nanoparticles. For the NW synthesis, a higher concentration of iron salts and a higher molecular weight dextran (MW 20,000 or 40,000, Sigma) were used. In one preparation 0.63 g of FeCl₃.6H₂O and 0.25 g FeCl₂.4H₂O were mixed with 4.5 g dextran in 10 mL of Millipore water at room temperature. This acidic solution was neutralized by the dropwise addition of 1 mL concentrated aqueous ammonia under vigorous stirring and a steady purge of nitrogen, and it was then heated at ˜70° C. for 1 hr. After purification by centrifuge filtering column (100,000MWCO, Millipore), the magnetic colloid was cross linked in strong base (5M aqueous NaOH solution) with epichlorohydrin (Sigma) and filtered through a 0.1 μm pore diameter membrane (Millipore). NW with a size range of 5080 nm were separated using a MACS® Midi magnetic separation column (Miltenyi Biotec). Nanosphere (NS) with a size range of 25˜35 nm were prepared using techniques known in the art. NW or NS with different numbers of free amines were prepared for peptide conjugation by reacting them with different concentrations of aqueous ammonia at room temperature for 48 hrs. The amine number per NS was measured using the SPDP assay. The amine number per NW was calculated assuming that the molecular weight of a NW is 7 times higher than a NS, based on the mean value of aggregated iron oxide cores for one NW observed in the TEM images and supported by the light scattering (DLS) data. Negatively charged NW (NW-N) were prepared by reacting non-aminated NW with 1 M chloroacetic acid in strong base (5M aqueous NaOH solution) for 2 hrs at room temperature. Micromod IO nanoparticles (50 nm nanomag-D-SPIO with amines) were obtained from Micromod Partikeltechnologie GmbH, Rostock, Germany. Feridex IO nanoparticles were obtained from Berlex, N.J., USA.

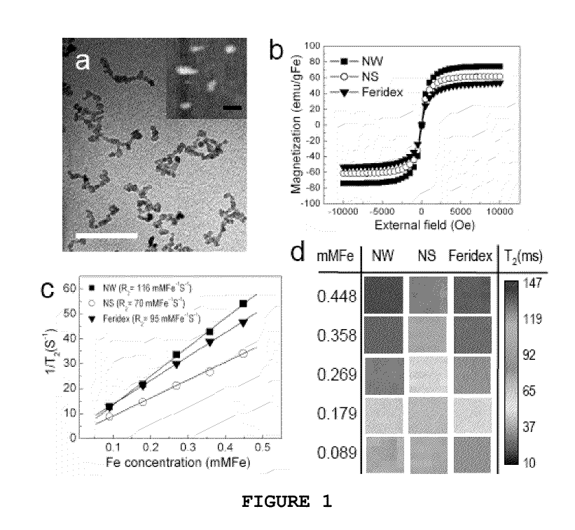

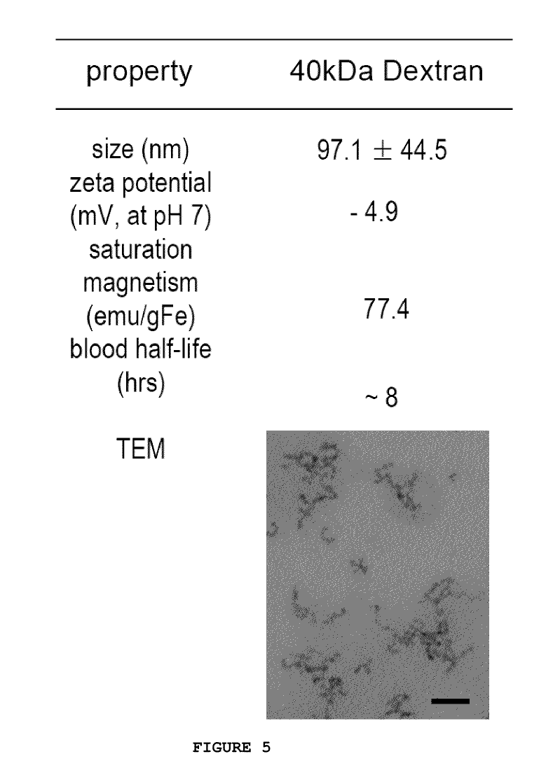

The nanostructure appears as linearly aggregated IO cores with a mean hydrodynamic size (in the long dimension) of 65 nm (FIG. 1a and Table 2). When a higher molecular mass dextran (40 kDa) was used branched IO cores were obtained, with a larger average size (˜100 nm) and a broader size distribution (FIG. 5). More than 80% of the nanostructures synthesized with 20-kDa dextran displayed chain-like shapes in the TEM images. This morphology did not appear to be due to a drying effect in the preparation of the TEM samples, as the particle sizes derived from the TEM images were well correlated with hydrodynamic diameter measurements by dynamic light scattering for both the NW and nanospheres (NS). In addition, NS synthesized using known techniques (designated here as CLIO, for Cross-Linked Iron Oxide) exhibit physical sizes and shapes, magnetic responses, and biological properties similar to what has been previously reported (FIG. 1, Table 2, and FIG. 6). NW show a slightly larger saturation magnetization value (74.2 emu/gFe vs. 61.5 emu/gFe and 53.5 emu/gFe) and a higher MR contrast (R₂=116 mMFe⁻¹S⁻¹ vs R₂=70 mMFe⁻¹S⁻¹ and R₂=95 mMFe⁻¹S⁻¹) than NS and Feridex (FIG. 1b). The one-dimensional assembly of magnetic nanocrystals in NW enhances the orientation of the magnetic moments of the nanoparticle constituents along the direction of the applied magnetic field, increasing the net magnetization. The increased MR contrast observed for NW is thought to be due to enhanced spin-spin relaxation of water molecules caused by the slightly larger magnetization value and one-dimensional assembly of magnetic nanoparticles.

TABLE 2
Characteristics of Amine-functionalized Magnetic
Iron Oxide Nanoworms and Nanospheres Studied^{a}

| | | | | | amine number | |
|---|---|---|---|---|---|---|
| | size | blood | R₂ | zeta potential | per gFe | per |
| sample^{b} | (nm)^{c} | T_{1/2} (min)^{d} | (mM Fe⁻¹S⁻¹)^{e} | (mV, at pH 7) | (×10²⁰) | NW/NS^{f} |
| NS | 30.3 ± 5.2 | 1060 | 70 | −5.1 | | |
| NS-7 | | 850 | | −1.8 | 3.6 | 7 |
| NS-30 | | 500 | | 6.7 | 15.7 | 30 |
| NS-59 | | <30 | | 17.2 | 30.9 | 59 |
| NW | 65.8 ± 8.9 | 990 | 116 | −5.3 | | |
| NW-42 | | 730 | | −2.2 | 3.2 | 42 |
| NW-P42 | | 1080 | | −2.8 | | 42 |
| NW-175 | | 460 | | 4.4 | 13.1 | 175 |
| NW-P175 | | 940 | | −2.3 | | 175 |
| NW-350 | | <30 | | 16.5 | 26.2 | 350 |
| NW-P350 | | 230 | | −0.8 | | 350 |
| NW-N | | 50 | | −20.3 | | |
| MM | 96.7 ± 16.1 | <30 | | | | ~500 |

^{a}None of the particles in this table contain targeting peptides.
^{b}NS = dextran-coated magnetic nanosphere. NW = dextran-coated magnetic nanoworm. NW-P = dextran-coated magnetic nanoworm with 5 kDa PEG linkers attached to the dextran. NW-N = dextran-coated magnetic nanoworm with pendant negatively charged carboxyl groups. MM = commercially obtained Micromod particles that have been aminated. The dextran coatings have been cross-linked and modified with free amines; the number after the letter identifier designates the approximate number of free amino groups per particle. For example, NW-P175 denotes a magnetic nanoworm with 175 amines to which PEG(5 kDa)-succinimidyl -methylbutanoate is conjugated.
^{c}Hydrodynamic size based on DLS measurement
^{d}Blood half-life (mouse, tail-vein injection) determined by magnetization or fluorescence measurement as described in the text. Relative error in these measurements is ±10%
^{e}R2 is longitudinal relaxation rate equal to reciprocal of T2 relaxation time (R₂ = 1/T₂) and is calculated with T2-weighted MRI map.
^{f}Number of peptides per single nanoworm or nanosphere.

Targeting peptide conjugation. One of two targeting peptides were used with the NW or NS samples: KDEPQRRSARLSAKPAPPKPEPKPKKAPAKK (F3) (SEQ ID NO:1), which preferentially binds to blood vessels and tumor cells in various tumors, and CREKA (SEQ ID NO:2), which recognizes clotted plasma proteins in the blood vessels and stroma of tumors. The fluorescein (FITC)-conjugated peptides were synthesized using Fmoc chemistry in a solid-phase synthesizer, and purified by preparative HPLC. Their sequence and composition were confirmed by mass spectrometry. For the F3 peptide, an extra cysteine residue was added to the N-terminus to allow conjugation with NW or NS. For near-infrared (NIR) fluorescence imaging, Cy7-labeled NW or NS were prepared by reacting aminated NW (500 μg Fe) or NS (900 μg Fe) in PBS buffer with 6 μg of Cy7-NHS ester (GE Healthcare Bio-Sciences) in DMSO (Sigma) for 1 hr to have same fluorescence per iron atom for both NW and NS (one Cy7 dye per one iron oxide core). The remaining free amines were used for conjugation with the targeting peptides. 500 μg Fe of Cy7-labed NW or NS were first reacted with 200 μg of Sulfosuccinimidyl-4-(N-maleimidomethyl)cyclohexane-1-carboxylate (sulfo-SMCC, Pierce Chemicals) or 2 mg NHS-PEG(5 kDa)-MAL (Nektar) in PBS solution for 1 hr and then purified using a desalting column (GE Healthcare Bio-Sciences). 200 μg of targeting peptide with a free terminal cysteine was then added to the 500 μg Fe NW or NS sample in PBS solution. After incubation for 2 h with mild shaking at room temperature, the sample was purified on a desalting column (GE Healthcare Bio-Sciences) for the CREKA samples or with a centrifuge filter (100,000 MWCO, Millipore) for the F3 samples, and then re-suspended in PBS solution. The FITC-peptide or Cy7 dye number per one NW or NS was determined with their absorbance spectra.

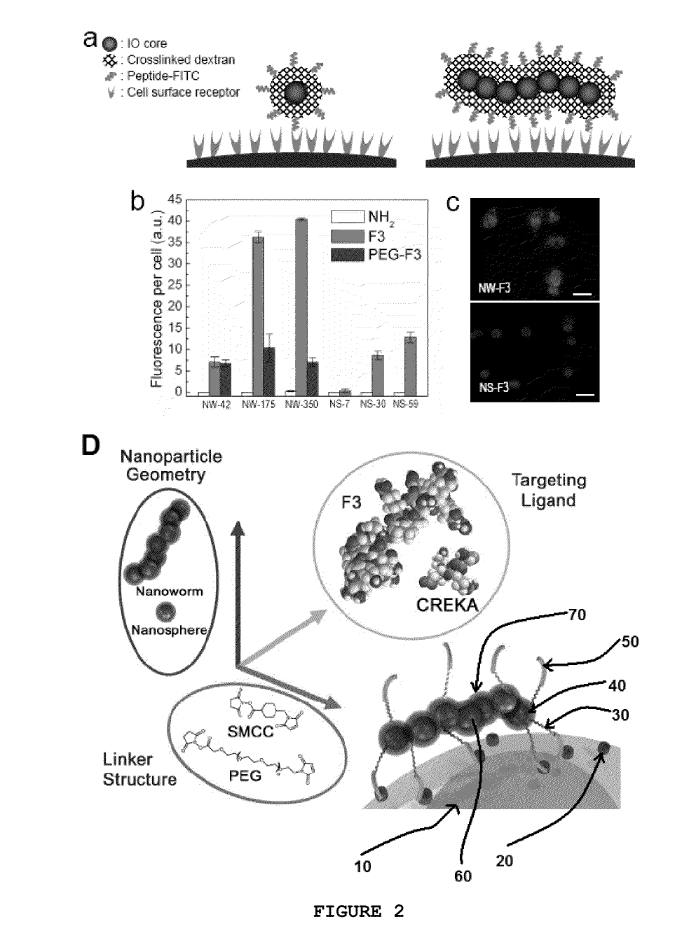

Conjugation of targeting peptides to magnetic nanoworms: in vitro cell internalization. The efficiency of peptide-targeted cellular internalization of NW compared with NS was tested in vitro. Conceptually, the elongated shape of the NW is expected to provide a larger number of interactions between the targeting ligands and their cell-surface receptors compared with spherical nanoparticles (FIG. 2a).

Magnetic measurement. A solution of the NW or NS sample was frozen and lyophilized to dryness in gelatin capsules. The capsules were inserted into the middle of transparent plastic straws. The measurements were performed at 298 K using a Quantum Design (CA, USA) MPMS2 superconducting quantum interference device (SQUID) magnetometer. The samples were exposed to direct current magnetic fields in stepwise increments up to one Tesla. Further corrections were made for the diamagnetic contribution of the capsule and straw.

Zeta potential measurements. Zeta potentials of NW or NS were measured using a Malvern (Worcestershire, UK) Zetasizer ZS90 equipped with an autotitration system. Zeta potentials were plotted in the pH range 3-9. The surface charge of the NW or NS samples is reported for the value of the zeta potential at pH 7 to simulate physiological conditions.

MRI T2 mapping. MRI T₂ mapping of NW or NS samples was performed using a 7 cm bore, Bruker (Karlsruhe, Germany) 4.7 T magnet. Samples were serially diluted with aqueous PBS (Mediatech) in a 384-well plate, containing 95 μl total sample/well.

In vitro cell internalization. MDA-MB-435 human breast carcinoma cells were maintained in Dulbecco's Modified Eagle's Medium (DMEM) supplemented with 10% fetal bovine serum (FBS) and 100 μg/ml penicillin-streptomycin. For cell internalization tests, the 10,000 cells were seeded into each well of 24-well plates and cultured overnight. The cells were then incubated with 40 μg (total Fe content) of Cy7-labeled peptide-conjugated NW or NS per well for 30 min, 1 h, or 2 h at 37° C. in the presence of 10% FBS (triplicate per NW or NS formulation). The wells were rinsed three times with cell media and then imaged in the Cy7 channel (762 nm excitation/800 nm emission) with a NIR fluorescent scanner (LI-COR biosciences). The relative fluorescence of the images (each well) was analyzed using the ImageJ (NIH) or OsiriX (Apple) programs. To quantify the internalized amount of NW or NS, the cells were carefully detached from each well using trypsin-EDTA, and centrifuged into a pellet. The pellets were freeze-dried in gelatin capsules, and then analyzed for magnetization using SQUID. For fluorescence microscopy, the cells (3000 cells per well) were seeded into 8-well chamber slides (Lab-Tek) overnight. The cells were then incubated with 10 μg (total Fe content) of Cy7-labeled peptide-conjugated NW or NS per well for 3 h at 37° C. in the presence of 10% FBS. After incubation, the slides were rinsed three times with PBS, fixed with 4% paraformaldehyde, and then washed three times with PBS and mounted in Vectashield Mounting Medium with 4′,6-diamidino-2-phenylindole (DAPI; Vector Laboratories, Burlingame, Calif.). The slides were observed with a fluorescence microscope (Nikon, Tokyo, Japan).

Cell internalization was used to probe the multivalent effect provided by the compositions and materials of the disclosure. An F3 peptide was used, which selectively targets cell surface nucleolin in tumor cells and tumor endothelial cells, and is known to have cell-penetrating properties. FITC-tagged F3 peptides were conjugated to dextran-coated, aminated NW or NS through the sulfhydryl group of a cysteine residue that had been added to the peptide. The peptides were conjugated via a short crosslinker (sulfo-SMCC) or a 5 kDa polyethylene glycol) (PEG) chain. Absorbance assays of the F3-conjugates showed that the numbers of peptides coupled to the particles could be controlled (Table 3). Additionally, F3 conjugation to the particles through PEG chains resulted in fewer peptides per particle.

TABLE 3
Loading of Targeting Peptides on Magnetic
Iron Oxide Nanoworms and Nanospheres^{a}

| | Number of F3 | | | Number of CREKA | |
|---|---|---|---|---|---|
| | peptides | | | peptides | |
| | per | per gFe | | per | per gFe |
| Sample^{b} | NW/NS^{c} | (×10²⁰) | Sample^{b} | NW/NS^{c} | (×10²⁰) |
| NS-7-F | 5 | 2.6 | NS-7-C | 6 | 3.1 |
| NS-30-F | 10 | 5.3 | NS-30-C | 18 | 9.4 |
| | | | NS-P30-C | 13 | 6.8 |
| NS-59-F | 12 | 6.3 | NS-59-C | 27 | 14.1 |
| NW-42-F | 23 | 1.7 | NW-42-C | 29 | 1.6 |
| NW-P42-F | 16 | 1.2 | NW-P42-C | 23 | 1.2 |
| NW-175-F | 69 | 5.1 | NW-175-C | 117 | 6.3 |
| NW-P175-F | 48 | 3. | NW-P175-C | 60 | 3.2 |
| NW-350-F | 83 | 6.2 | NW-350-C | 205 | 10.2 |
| NW-P350-F | 59 | 4.4 | NW-P350-C | 90 | 4.9 |
| | | | MM-500-C | 350 | |

^{a}The particles in this table contain targeting peptides.
^{b}Sample numbers are as defined in Table 2. The -F or -C suffix denotes F3 or CREKA-conjugated particle, respectively. These targeting peptides have been conjugated to the free amines on the dextran coatings via sulfo-SMCC crosslinker or via PEG crosslinker. F3 peptide selectively targets cell surface nucleolin in tumor cells and tumor endothelial cells, and CREKA peptide recognizes clotted plasma proteins, which accumulate in tumors but not in normal tissues.
^{c}Number of peptides per single nanoworm or nanosphere.

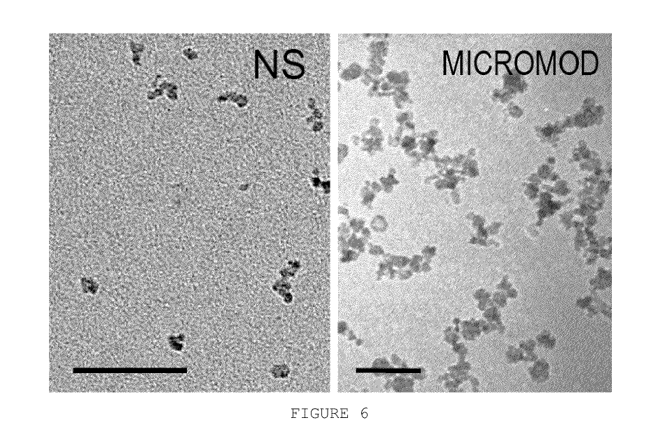

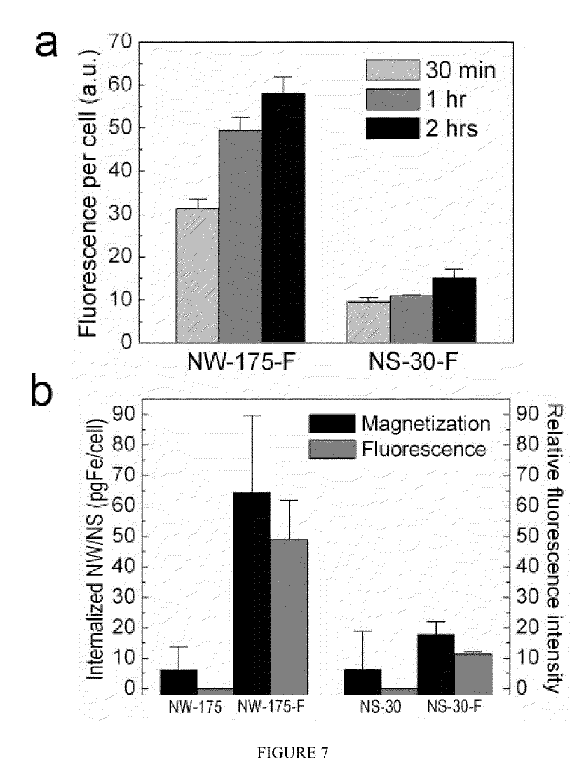

Fluorescently labeled (Cy7) NW and NS were used to determine the efficiency of cell internalization. The total number of attached Cy7 dye molecules was controlled to yield the same fluorescence intensity on a per-iron basis for both types of particles. The various formulations of Cy7-labeled, F3-conjugated particles were incubated with MDA-MB-435 human breast cancer cells for 2 hrs. The cells were imaged with a NIR fluorescence scanner. The nanoworms NW-42-F, NW-175-F, and NW-350-F display similar numbers of F3 targeting peptides per iron atom compared with NS-7-F, NS-30-F, and NS-59-F, respectively (Table 3). No internalization of NS or NW into the tumor cells was seen without F3 coating of the particles, and internalization increased with the number of F3 peptides attached per NW/NS (FIG. 2b; FIG. 7), and internalization of NW was significantly enhanced relative to NS on a per-iron basis. The time-dependence of the internalization also supports the postulated importance of multivalent interactions in particle endocytosis (FIG. 7). Overall, the data show that NW provide a more efficient means to bring a set quantity of iron oxide into a cell.

The SQUID (Superconducting Quantum Interference Device) magnetometry provides a direct measure of the total number of magnetic IO nanoparticles in a sample, as it measures the magnetization of a sample rather than the total iron content or the fluorescence intensity from a molecular tag. The SQUID measurements are relevant to the MRI imaging applications, because the magnetization data correlates with T₂. The SQUID technique has the additional advantage that it can be performed on cells, cell extracts, or on whole organs, and little sample workup is needed. The SQUID data confirmed that NW were more effectively taken up by the cells; for F3-conjugated particles incubated for 2 h prior to analysis, ˜65 pg of Fe from NW was internalized per cell, whereas only ˜16 pg of Fe was internalized per cell from NS, (samples NW-175-F and NS-30-F, FIG. 7). These magnetization data confirm the fluorescence data obtained using Cy7-labeled F3-conjugated NW and NS. In microscope images, fluorescence from F3(FITC)-conjugated NW is significantly more intense than from F3(FITC)-conjugated NS after 3 hrs of incubation in the cytoplasm (FIG. 2c). These results indicate that NW with a larger number of attached targeting ligands bind to cells with a higher avidity and move into the cytoplasm more rapidly than NS. Additionally, since immunogenicity of targeting ligands are of concern for in vivo imaging and therapeutic applications, it is important that the total number of peptides per iron atom to achieve efficient targeting is much less for NW than NS.

Short PEG chains are often used to avoid MPS uptake minimizing interactions of blood proteins with nanomaterials. Using a PEG linker to attach the F3 peptide to the NW resulted in less cellular uptake, even when a large number of F3 targeting peptides where attached to the particles (NW-P175-F and NW-P350-F, FIG. 2b).

In vivo behavior of peptide-conjugated magnetic nanoworms. Circulation in the blood stream for a long period of time is a factor for in vivo target-specific reporting and drug delivery with nanomaterials. In vivo circulation of unmodified NW using doses of 3 mg Fe/kg and 10 mg Fe/kg body mass was tested in mouse.

Blood half-life and biodistribution. To quantify the in vivo circulation times of NW or NS samples in Nude BALB/c mice (n=3-4 for each formulation), heparinized capillary tubes (Fisher) were used to draw 15 μL (for fluorescence) or 70 μl (for magnetization) of blood from the periorbital plexus at different times after intravenous injection of the NW or NS samples (1, 3, or 10 mg Fe/kg body mass). The extracted blood samples were immediately mixed with 10 mM EDTA to prevent coagulation. For Cy7-labeled NW or NS formulations, blood extracted at different times was imaged in a 96-well plate in Cy7 channel (762 nm excitation/800 nm emission) with a NIR fluorescence scanner (LI-COR biosciences, NE, USA). The images were analyzed using the ImageJ (NIH) or Osirix (Apple) programs. For non-labeled NW or NS samples, blood samples extracted at different times were immediately freeze-dried in gelatin capsules, and then analyzed for magnetization using SQUID. The blood half-life was calculated by fitting the blood fluorescence or magnetization data to a single-exponential equation used in a one-compartment open pharmacokinetic model. Additionally, the NW were extracted from the blood stream 24 hrs after intravenous injection and rinsed completely 5 times on a magnetic column (Miltenyi Biotec) with PBS solution, and their size was analyzed using DLS. For the mouse biodistribution studies, unmodified NW in PBS (100 μL) were intravenously injected into Nude BALB/c mice at a dose of 3 mg Fe/kg body mass (n=3 for both the PBS controls and the NW samples). The animals were sacrificed 24 hrs after injection by cardiac perfusion with PBS under anesthesia, and the blood, brain, heart, kidney, liver, lung, lymph node, skin and spleen were collected. Organs and blood were immediately weighed, freeze-dried in gelatin capsules, and then analyzed for magnetization using SQUID.

In vivo tumor homing. MDA-MB-435 human breast carcinoma cells or HT1080 human fibrosarcoma cells (1×10⁶) were injected into the mammary fat pad or subcutaneously injected into Nude BALB/c mice, respectively. Tumors were used when they reached ˜0.5 cm in size. Some 0.2 cm or 1 cm tumors were used to compare the dependence of tumor size on NW homing. Cy7-labed or non-labeled NW or NS were intravenously injected into mice (n=3˜8 for each formulation) with a dose of 1 mg Fe/kg body mass (for fluorescence studies) and 3 mg Fe/kg body mass (for magnetization studies). For real-time observation of tumor/liver uptake, animals were imaged under anesthesia in Cy7 channel using the BonSai fluorescence-imaging system (Siemens, Pa., USA) 6 hrs, 24 hrs or 48 hrs after injection. For NIR fluorescence imaging of organs, animals were sacrificed 24 hrs after the injection by cardiac perfusion with PBS under anesthesia, and organs were dissected and imaged in Cy7 channel with a NIR fluorescence scanner (LI-COR biosciences, NE, USA). All the NIR images for animals or organs were taken at the same exposure time. To quantify the amount of NW or NS homing, collected tumors were weighed, freeze-dried in gelatin capsules, and then analyzed for magnetization using SQUID. For histologic analysis, frozen sections of tumors were prepared. The sections were fixed with 4% paraformaldehyde and stained with DAPI for observation of NW or NS only. The rat anti-mouse CD-31 (1:50, BD PharMingen) and biotinylated mouse fibrin(ogen) antiserum (1:50, Nordic) were used for immunochemicostaining of tumor tissue sections. The corresponding secondary antibodies were added and incubated for 1 hour at room temperature: AlexaFluor-594 goat anti-rat or rabbit IgG (1:1,000; Molecular Probes), streptavidin Alexa Fluor 594 (1:1000; Molecular Probes). The slides were washed three times with PBS and mounted in Vectashield Mounting Medium with DAPI. At least three images from representative microscopic fields were analyzed for each tumor sample.

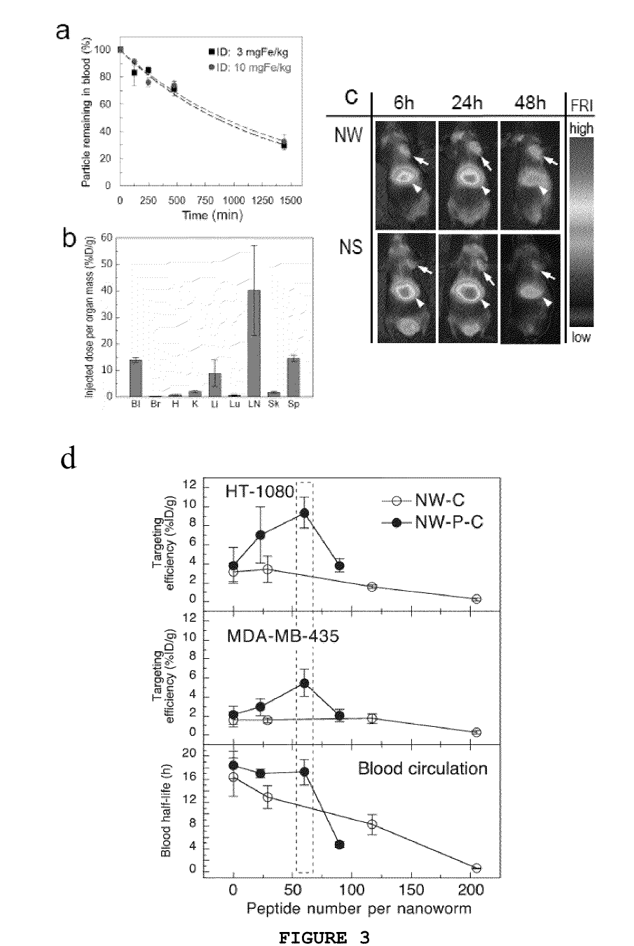

For both injection doses, the NW exhibited long circulation times (FIG. 3a and Table 3). NW extracted from the blood stream 24 hrs after intravenous injection showed a slight increase in size (from ˜65 to ˜80 nm by DLS), presumably attributable to protein binding during the circulation. The in vivo circulation half-life was dependent on the number of surface amine groups and the surface charge on the NW (Table 3). As the number of surface amine groups and hence the net particle charge increases, the in vivo circulation time decreases. Free surface amines may attract certain plasma proteins related to opsonization, as maintenance of a surface charge of ±5 mV (zeta potential at pH 7) after surface modification seems to be required to achieve in vivo blood half-life in excess of 8 hrs. The samples NW-42 (˜42 free amine groups per nanoworm) and NS-7 (˜7 free amine groups per nanosphere), NW-175 and NS-30, and NW-350 and NS-59 can be compared with each other, respectively, since each of these pairs has similar total number of free amine groups per iron atom and display similar in vivo circulation half-lives (Table 2). NW with no peptide coating display blood half-lives similar to those of non-coated NS even though the NW are larger than the NS (˜65 vs ˜30 nm by DLS, respectively). Attachment of PEG linkers to aminated NW (e.g. NW-P175 or NW-P350, Table 2) improves the circulation time, presumably by neutralizing the positive surface charge and reducing the binding of plasma proteins involved in opsonization.

The biodistribution of NW 24 hrs post injection was similar to that reported previously for CLIO. These particles both display a tendency to undergo MPS clearance in the liver, spleen, and lymph nodes (FIG. 3b). However, there are some differences in the biodistribution of NW relative to CLIO. The fraction of injected particles in the kidney relative to the liver is lower for NW compared with CLIO, whereas the spleen:liver particle concentration ratio is higher for NW.

The NW passively accumulate in tumors, and they appear to display long residence times once they get in. The reason is believed to be that tumor vessels are generally found to be more permeable to nanoparticles than the vessels of healthy tissues. To test the role of this passive tumor targeting, intravenously injected mice bearing MDA-MB-435 tumors with Cy7-labeled NW or NS (1 mg Fe/kg) were imaged at various intervals after injection with an NIR fluorescence-imaging system. Passive tumor uptake of NW was slightly greater than NS, although the difference was not statistically significant (FIG. 3d and FIG. 8). The data were supported by ex-vivo SQUID analysis of both MDA-MB-435 HT1080 (highly vascularized tumor model) tumors (FIG. 8). Some NS label was observed in the bladder 6 hrs and 24 hrs after intravenous injection, suggesting more effective kidney clearance of these smaller nanostructures (FIG. 3d). Interestingly, the NW remain in the tumor even 48 hrs after injection, whereas the NS are almost completely eliminated within this time period. The rapid clearance of NS from these tumors is similar to what was observed in previous in vivo studies of RGD-conjugated semiconductor quantum dots. The data indicate that once NW extravasate into tumor tissue from the blood vessels, they become physically trapped and do not readily re-enter the blood stream. This result suggests that more effective diagnostic imaging or drug delivery may be possible with NW than with NS.

The efficiency of NW and NS in homing peptide-directed targeting into tumors was analyzed. Two tumor-homing peptides were used to target the particles: F3 and a pentapeptide with the sequence CREKA. The CREKA peptide recognizes clotted plasma proteins, which accumulate in tumors but not in normal tissues. CREKA-conjugated 10 nanoparticles accumulate in tumors, but do so effectively only after pre-injection with Ni-liposomes designed to inhibit MPS uptake. This inability of nanoparticles to evade the MPS poses a significant limitation to nanoparticle targeting in vivo. To test the susceptibility of peptide-conjugated NW to MPS uptake, NW preparations containing different numbers of peptides, different degrees of amination, and the peptide conjugated either through a short linker or PEG (Table 2) were examined. The MDA-MB-435 human breast cancer xenograft and HT1080 human fibrosarcoma xenograft tumor models were chosen for these studies.

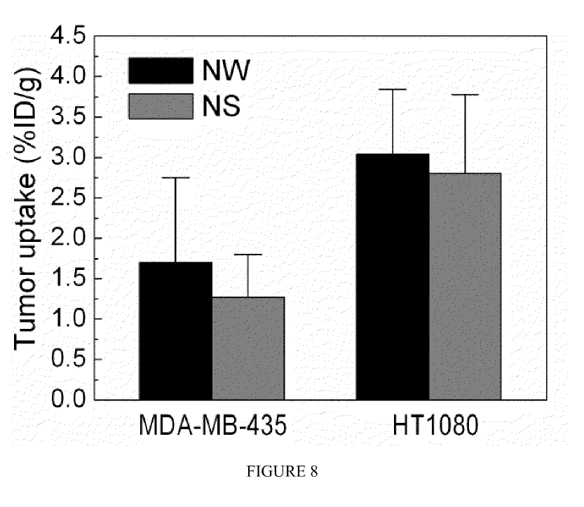

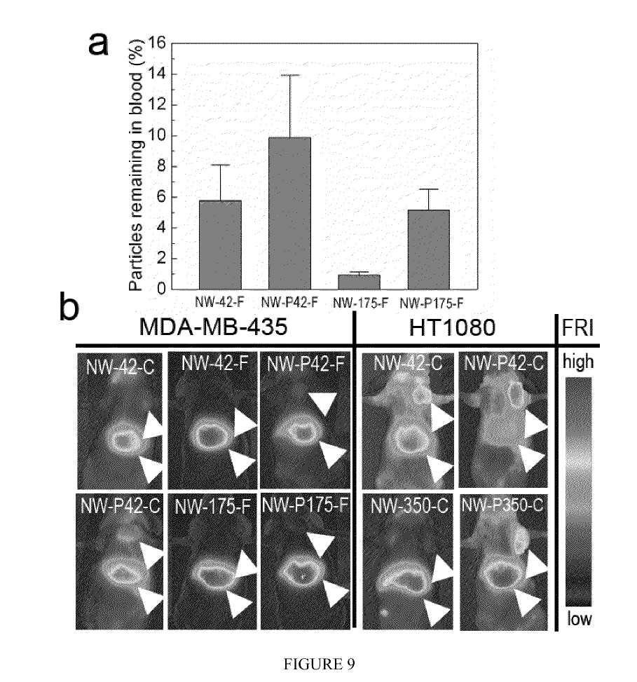

The in vivo targeting capability of Cy7-labeled, F3-conjugated NW (NW-F) in mice bearing MDA-MB-435 tumors was tested. The NW-F preparations were essentially cleared by the liver from the blood within 30 min of intravenous injection, regardless of the peptide number or the presence of a PEG layer (FIG. 9). F3 is a 31-amino-acid sequence with large number of positive residues; this excess charge is presumably responsible for the short circulation time of the particles conjugated with this peptide. The fluorescence imaging experiments using NW-F show no tumor homing detectable by whole body imaging (FIG. 9b), whereas previous studies have documented F3-directed tumor homing with other types of particles and using more sensitive detection methods.

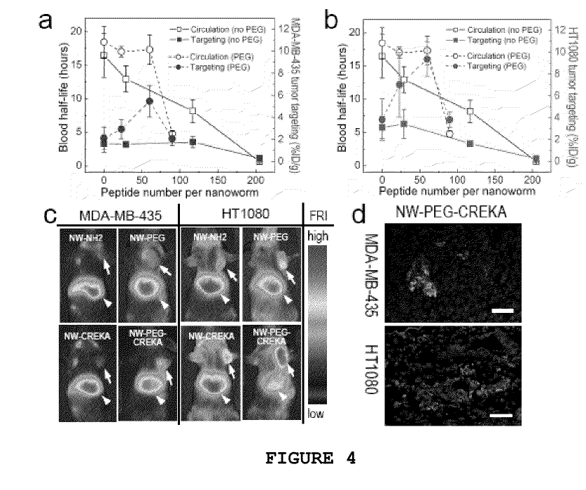

Next, the ability of CREKA-conjugated NW (NW-C) to home to tumor targets in vivo was tested. The NW-C preparations that maintained the longest circulation times contain ≦60 CREKA homing peptides per NW and a PEG layer (FIGS. 4a and 4b). Nanoparticles containing thousands of attached CREKA molecules along with pre-injection of Ni liposomes have been shown to be effective at binding to clotted proteins and in tumor homing. However, for the NW, in vivo targeting efficiency diminished as the number of CREKA molecules increased past 60 per NW (FIG. 4a, 4b and Table 2). This significant decrease is attributed to in vivo circulation reduced by the presence of unreacted amines or the dextran coating damaged during the amination step; these chemical groups or the iron oxide cores exposed by weakening a bonding between dextran coating and iron oxide cores are expected to interact with plasma proteins involved in opsonization. CREKA conjugation significantly increased the in vivo tumor targeting efficiency of PEGylated NW in both tumor types studied, with greater efficiency observed in the HT1080 tumors (FIG. 4a, 4b and FIG. 9b). Consistent with the short blood half-life, PEGylated NW conjugated with the largest number of CREKA homing peptides (NW-P350-C) exhibited lower tumor uptake in each tumor model than less densely conjugated particles (FIG. 4a, 4b and FIG. 9b). In addition, CREKA-conjugated NW without PEG did not show significant tumor uptake in either tumor type. PEGylated CREKA-conjugated NW not only prolongs the in vivo circulation, but may also facilitate the binding of CREKA to the blood clots due to free movement of the peptide when linked via longer PEG chain as opposed to short crosslinker (Sulfo-SMCC).

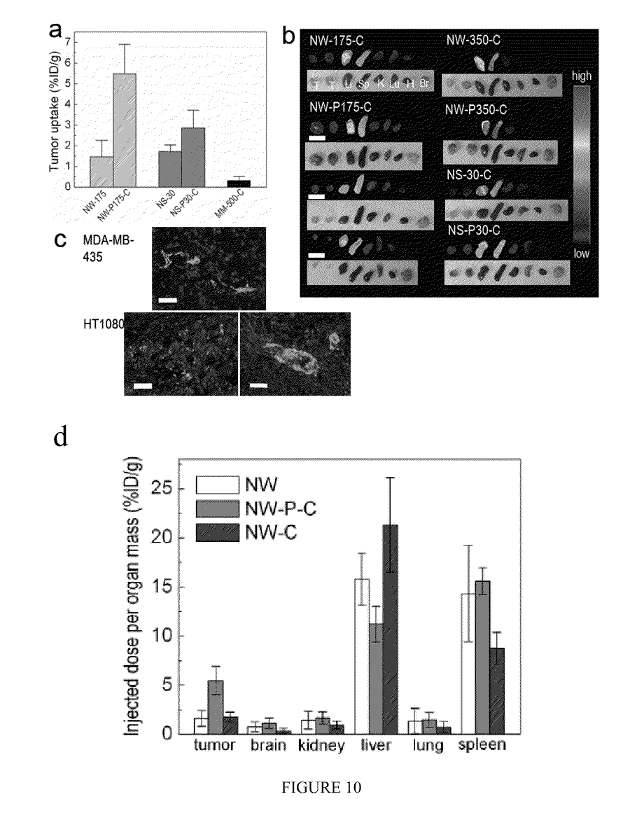

CREKA-conjugated NW displayed somewhat improved uptake in MDA-MB-435 tumors compared with CREKA-conjugated NS. (FIGS. 10a and 10B). Both the NW and NS samples used in this study perform better than CREKA-modified IO nanoparticles obtained from a commercial source (Micromod) that were the subject of a previous study. In that study, prominent tumor homing was achieved only by employing decoy particles to circumvent liver uptake, whereas the NW in the present study homed to tumors without such intervention. NIR fluorescence microscope images of mice injected with Cy7-labeled CREKA-conjugated NW confirm the tumor uptake results obtained by SQUID (FIG. 4c and FIG. 9b). Here, PEGylated CREKA-conjugated NW (sample NW-P175-C) in both tumors display significant increases in the tumor:liver ratio compared with the other samples studied. Importantly, targeting of PEGylated CREKA-conjugated NW was also observed in smaller tumors (size 0.2 cm, FIG. 10b). NIR fluorescence images of the organs collected 24 hrs post-injection reveal that most of the NW are in the liver and spleen (indicative of MPS clearance) whereas small amounts are observed in the kidney, lung, and heart (FIG. 10b).

Histological analysis showed that most of the PEGylated CREKA-conjugated NW colocalize with large blood vessels in the MDA-MB-435 tumor, whereas most of the NW in the HT1080 tumor appeared to have extravasated into the tumor tissue along the smaller vessels (FIG. 4d). In addition, NW in the MDA-MB-435 tumor colocalized with fibrin(ogen) in the blood vessels, indicative of self-amplifying homing that has been observed previously (FIG. 10c). NW in the HT1080 tumor colocalized along fibrin(ogen) in the tumor stroma (left panel in FIG. 10c), as well as with fibrin(ogen) in blood vessels (right panel). These results suggest that the HT1080 tumors, like other tumors, contain clotted plasma proteins that provide initial binding sites for the CREKA peptide, and that the nanoparticles induce additional clotting within the tumor. Thus, the uptake of CREKA-conjugated NW in the HT1080 tumor is due to passive transport across leaky blood vessels, active peptide-mediated binding, and self-amplifying homing due to clotting induced by the CREKA-coated particles. Overall, these results indicate that long-circulating NW, with the appropriate surface charge and number of targeting ligands, can provide improved in vivo tumor targeting.

In contrast to the in vivo behavior of F3-modified NW, when CREKA is used as the targeting peptide (NW-C), the NW effectively home to their tumor targets. CREKA is a short linear peptide that is neutrally charged and most likely non-immunogenic. A tradeoff between the number of attached peptides and the efficiency of tumor targeting is observed for the NW-C preparations; the most effective in vivo tumor targeting is observed with ˜60 CREKA peptides per NW. This number correlates with a substantial decrease in blood half-life that is observed when >60 CREKA peptides are attached to a NW. The trend is observed for both HT1080 and MDA-MB-435 tumor types, although the overall targeting efficiency of NW-C is greater for HT1080 tumors. Additionally, in contrast to the NW-F preparations, significantly long circulation times (>10 h) are observed with some of the NW-C preparations.

For both HT1080 and MDA-MB-435 tumors, greater targeting efficiency is observed for nanoworms comprising CREKA (NW-C) when a PEG linker is used to attach the CREKA targeting group. It is postulated that the PEG linker facilitates CREKA homing by providing a less restrictive environment (relative to the short sulfo-SMCC linker), improving the peptide's ability to bind to clotted plasma proteins associated with the tumor. Additionally, the PEG linker increases residence time of the nanostructure in the blood stream.

The decrease in circulation time observed for NW containing >60 CREKA peptides is possibly attributable to the presence of unreacted amines and damage to the dextran coating (exposing bare IO cores) that occurs during preparation of the more extensively functionalized nanoparticles. The data indicate that the blood half-life of a targeting molecule/nanoparticle ensemble must be considered when selecting the appropriate ligand to target a tumor. As also observed with NW-F, a dramatic decrease in circulation time and a corresponding decrease in targeting efficiency can occur when targeting ligands are linked to nanomaterials.

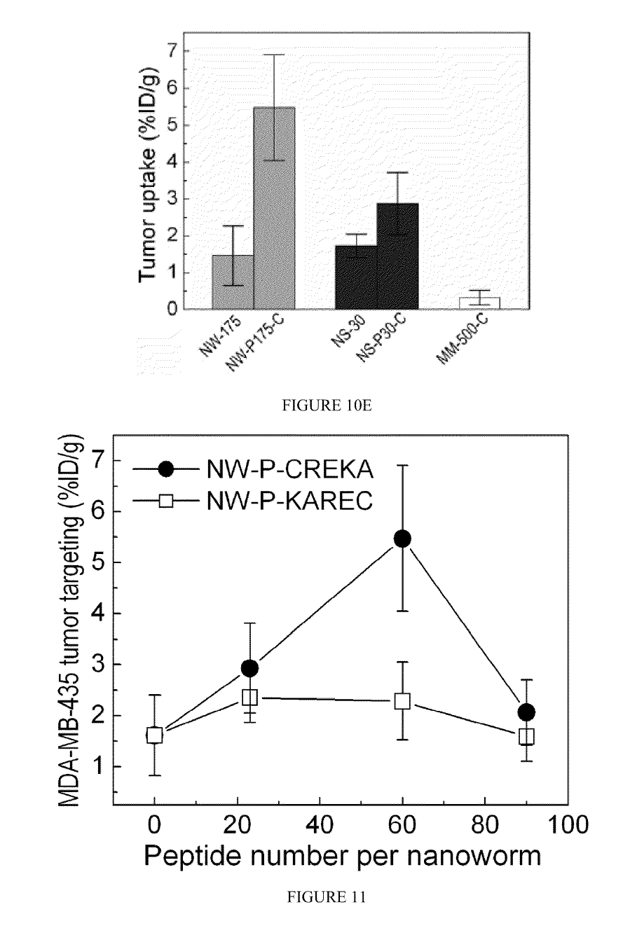

A control experiment using KAREC, a scrambled version of CREKA, was performed in mice bearing MDA-MB-435 tumors. KAREC was attached to the NW using a PEG linker, and the formulations displayed similar circulation times to the PEGylated NW-C formulations. Significantly lower tumor targeting efficiency was observed with the scrambled peptide (FIG. 11).

NIR fluorescence images of mice injected with NW-C confirm the tumor uptake results obtained by magnetic (SQUID) measurements (FIG. 4). Significant increases in the tumor:liver fluorescence signal ratio are observed in both tumor types for PEGylated NW-C (sample NW-P175-C) compared with the other samples studied. Targeting of PEGylated NW-C could be observed in smaller tumors (size 0.2 cm, see FIG. 10b), indicating that the formulation is applicable for the detection of tumors at the early stages of growth. NIR fluorescence images of organs and biodistribution results in mice bearing MDA-MB-435 tumors 24 h post-injection reveal that most of the NW-C are cleared by the liver and the spleen of the mouse, similar to what is observed with other targeted nanomaterials (FIG. 10d). PEGylated NW-C show relatively greater uptake by the spleen, while NW-C formulations containing the short chain linker display somewhat greater uptake by the liver.

Histological analysis revealed that most of the PEGylated NW-C localize with large blood vessels in the MDA-MB-435 tumor, whereas they extravasate into the tumor tissue along the smaller vessels in the HT1080 tumor. In addition, NW in the MDA-MB-435 tumor colocalize with fibrin(ogen) in the blood vessels, indicative of the self-amplifying homing. NW in the HT1080 tumor localize with fibrin(ogen) in blood vessels as well as in tumor stroma. These results suggest that HT1080 tumors, like other tumors contain clotted plasma proteins that provide initial binding sites for the CREKA peptide, and that the nanoparticles induce additional clotting within the tumor. Thus, the larger uptake of NW-C observed in the HT1080 tumor relative to MDA-MB-435 tumor is attributed to passive transport across a highly vascularized and porous microstructure, active peptide-mediated binding, and self-amplifying homing due to clotting induced by the CREKA-coated particles.

NW are more effectively attach to tumor cells in vitro while exhibiting comparable blood circulation times relative to spherical NS. The superior in vitro targeting efficiency was attributed to multivalent interactions between the elongated NW and receptors on the tumor cell surface. Similar improvement were seen in tumor targeting by NW in vivo. The optimized NW-C formulation (NW-P175-C) displays significantly higher levels of uptake in MDA-MB-435 tumors relative to NS-C (NS-P30-C). Targeting efficacy was also compared between NW-C and CREKA-conjugated commercial 10 nanoparticles (MM-500-C, blood half-life: ˜30 min). This inability of the nanoparticles to evade the MPS by themselves highlighted a significant limitation to the practical application of nanoparticle therapies that is overcome using the elongated nanostructures of the disclosure.

### Example 2

Preparation of gold nanorod, magnetic nanoworm, and doxorubicin liposomes. Gold nanorods (NRs) were purchased from Nanopartz with a peak plasmon resonance at 800 nm and coated with polyethelene glycol (PEG) molecules [HS-PEG(5k)]. Superparamagnetic, dextran-coated iron oxide nanoworms (NWs) with a longitudinal size of ˜70 nm were synthesized, and derivatized with near-infrared (NIR) fluorophore, Cy5.5-NHS. For control NWs, partially Cy5.5-labeled aminated NWs were coated with a PEG molecule [NHS-PEG(5k)]. For LyP1-conjugated NWs (LyP1NWs), LyP1 peptides with extra cysteine were attached to partially Cy5.5-labeled aminated NWs via a PEG crosslinker [NHS-PEG(5k)-MAL]. Control liposomes (LPs), with no functional group were prepared from hydrogenated soy sn-glycero-3-phosphocholine (HSPC), cholesterol, and 1,2-distearoyl-snglycero-3-phosphoethanolamine-N-polyethylene glycol 2000 [DSPE-PEG(2k)] (75:50:6 mol ratio) by lipid film hydration and membrane (100 nm) extrusion. Incorporation of DOX was achieved using the pH gradient-driven protocol. For LyP1-conjugated LPs (LyP1LPs), LPs with maleimide groups were prepared from HSPC, cholesterol, DSPE-PEG(2k), and DSPE-PEG(2k)-MAL (75:50:6:6 mol ratio). LyP1 peptides with an extra cysteine were attached to maleimide-terminated LPs in PBS. LPs were intravenously injected in vivo to ensure control LPs and LyP1LPs exhibited similar circulation times (blood half-lives for both: ˜3 hrs).

In vitro cellular fluorescence imaging. The cells were treated with 80 ug Fe/mL of Cy5.5 labeled control NWs or LyP1NWs per well for 20 min at 37° C. or 45° C. in the presence of 10% FBS and incubated for an additional 2 hr at 37° C. in the presence of 10% FBS. The cells were then rinsed three times with cell medium, fixed, stained, and imaged by fluorescence microscopy.

In vivo temperature-induced tumor targeting of magnetic nanoworms. Mice bearing bilateral MDA-MB-435 human carcinoma tumors were intravenously injected with Cy7-labeled LyP1NWs or NWs and one flank of the mouse (containing one of the tumors) was immediately heated at 45° C. for 30 min in a temperature-controlled water bath. At 24 hrs post-injection, the tissues were harvested and the Cy7 fluorescence in tissues were imaged using NIR fluorescence imaging system (LI-COR Odyssey).

In vitro temperature-induced cytotoxicty of therapeutic nanoparticles. Cells were treated with free DOX, control LPs, or LyP1LPs with different concentrations at 37° C. or 45° C. for 20 min (in cell incubator) and then incubated for an additional 4 hrs at 37° C. The cells were rinsed with cell medium three times, and then further incubated for 44 hrs at 37° C. The cytotoxicity of free DOX, control LPs, or LyP1LPs was evaluated using MTT assay (Invitrogen). Cell viability was expressed as the percentage of viable cells compared to controls (cells treated with PBS).

In vivo tumor targeting of therapeutic nanoparticles by NR-mediated photothermal heating. Mice bearing bilateral MDA-MB-435 human carcinoma tumors were intravenously injected with NRs (10 mg Au/kg). At 72 hrs post-injection of NR, control LPs or LyP1LPs (3 mg DOX/kg) were systemically administered and the tumor in one flank was irradiated with NIR-light (˜0.75 W/cm² and 810 nm) for 30 min, maintaining an average tumor surface temperature at ˜45° C. under infrared thermographic observation. At 24 hrs post-injection of liposomes, doxorubicin fluorescence in the homogenized tumors was analyzed.

In vivo therapeutic studies. To study the effect of photothermal treatment on tumor volumes, mice bearing bilateral MDA-MB-435 human carcinoma tumors were intravenously injected with NRs (10 mgAu/kg). At 72 hrs post-injection of NR, control LPs, or LyP1LPs (3 mg DOX/kg) were systemically administered and the tumor in one flank was irradiated with NIR-light (˜0.70 or 0.75 W/cm² and 810 nm) for 30 min, maintaining average tumor surface temperature at 45° C. Each therapeutic cohort included 4˜6 mice. Tumor volume and mouse mass was measured every 3 days after the single treatment for a period of 3-4 weeks by an investigator blinded to the treatments administered. Survival rates (Kaplan Meier analyses) for the photothermal treatments were quantified using mice bearing single MDA-MB-435 human carcinoma tumors, intravenously injected with NRs (10 mgAu/kg). Control LPs or LyP1LPs (3 mg DOX/kg) were systemically administered 72 hrs post-injection and one of the tumor-bearing flanks was irradiated with NIR-light (˜0.75 W/cm² and 810 nm) for 30 min, maintaining average tumor surface temperature at ˜45° C. Each therapeutic cohort included 6 mice. Tumor volume and mouse mass was measured every 3 days after the single treatment for a period of 9 weeks by an investigator blinded to the treatments administered. Mice were sacrificed when tumors exceeded 500 mm³. Student's t test was used for statistical analysis of the results.

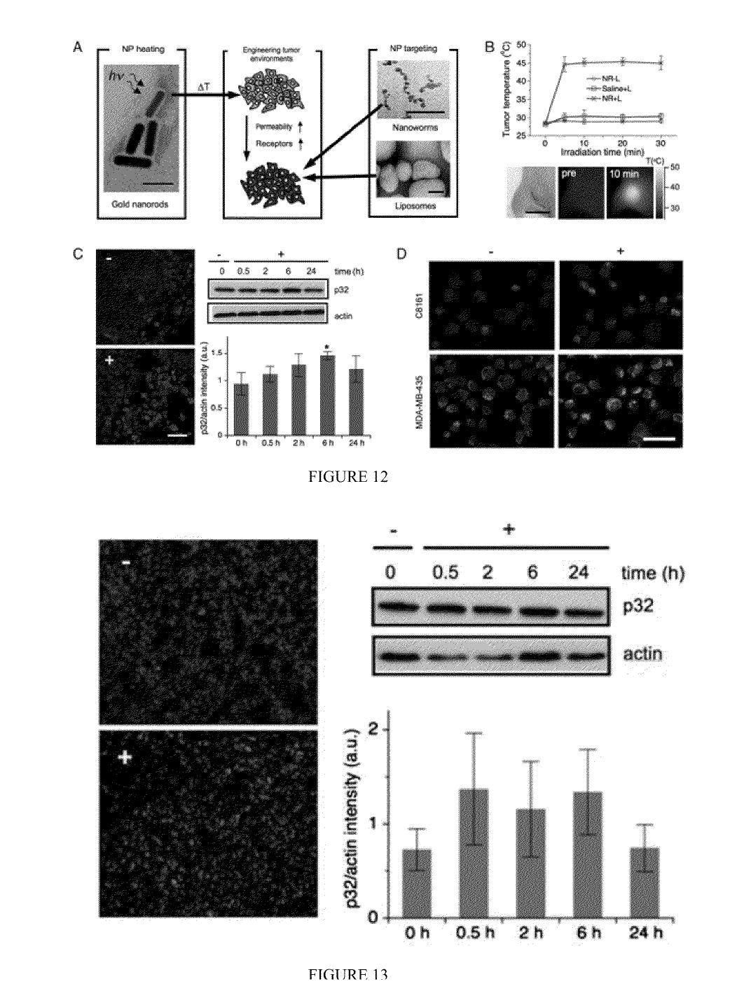

In a first stage of the cooperative nanoparticle system, the photothermally-heated gold nanorods are administered. Polyethylene glycol (PEG)-coated NRs with a maximum optical absorption of 800 nm are found to accumulate passively in a MDA-MB-435 xenograft tumor. Effective in vivo photothermal heating of the tumor is achieved by application of NIR irradiation (810 nm, ˜0.75 W/cm²) from a diode laser (FIG. 12b).

A cyclic nine-amino acid peptide (Cys-Gly-Asn-Lys-Arg-Thr-Arg-Gly-Cys (SEQ ID NO:3), referred to as LyP-1, was chosen as the targeting ligand based on a screen of several tumor targeting peptides in MDA-MB-435 xenograft tumors, which showed enhanced LyP1 accumulation in the heated tumors. The LyP-1 peptide has been reported to selectively recognize lymphatics and tumor cells in certain tumor types and subsequently inhibit tumor growth. Recently, it was found that the p32 or gC1qR receptor, whose expression is elevated on the surface of tumor-associated cells undergoing stress, is the target molecule for the LyP-1peptide. Thus, the targeting of LyP-1 was investigated as it relates to up regulation of p32 receptors in the heated tumor.

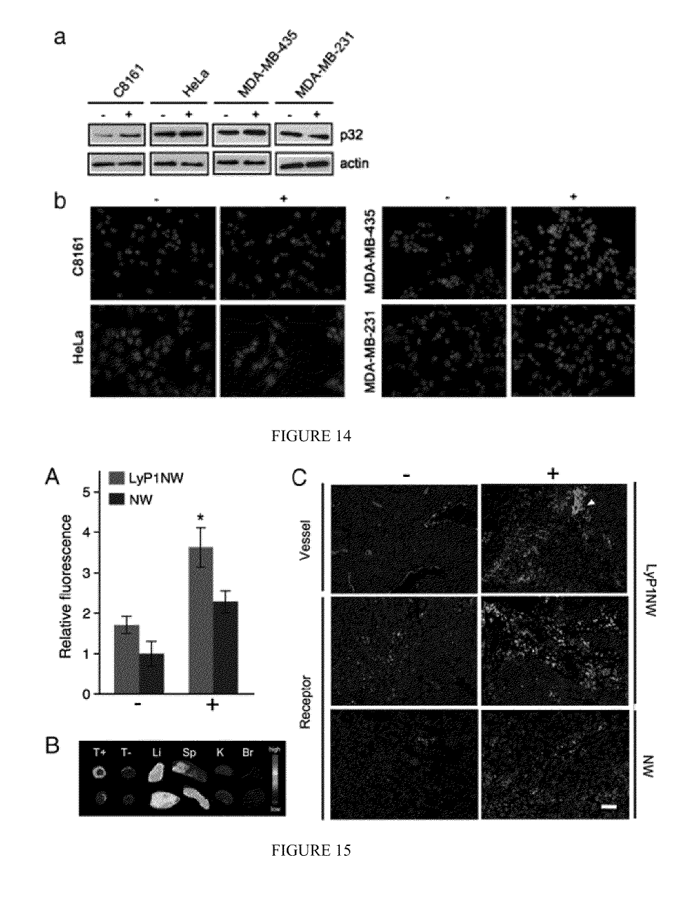

The level of p32 expression in MDA-MB-435 xenografts was examined as a function of time post-heat treatment. An externally measured temperature of 45° C. was chosen for the laser heat treatment based on a preliminary screen of temperature dependent nanoparticle accumulation. It has been reported that cancer cells are most vulnerable to hyperthermia, chemotherapeutics or a combined therapy above temperatures of 43° C. Expression of p32 on the MDA-MD-435 tumors was slightly up regulated 6 hrs after heat treatment, which then returned to almost normal levels 24 hrs post-treatment (FIG. 12c). Compared with the MDA-MB-435 tumors, less significant changes in the level of heat-mediated p32 expression were observed on C8161 tumors, known as the tumor type that expresses a considerably less amount of p32 compared to MDA-MB-435 tumor, over a 24 hr period post-heating (FIG. 13). Expression of p32 in cultured cells upon heat treatment exhibited a pattern similar to the in vivo xenograft results; the extent of p32 expression on C8161 cells (and cell surfaces) was less than that observed with MDA-MB-435 cells (FIG. 14).

The interaction of nanoparticles decorated with LyP-1 peptides with cancer cells was then examined upon heat treatment. An optimized formulation of NWs was prepared, and coated with LyP-1 peptides via PEG linkers (˜40 peptides per nanoworm). Significant quantities of the LyP-1 peptide-conjugated NWs (LyP1NWs) were internalized into heated MDA-MB-435 cells relative to unheated cells. In contrast, the C8161 cells displayed lower heat-mediated internalization than the MDA-MB-435 cells (FIG. 12d). The colocalization of p32 receptors and LyP1NW was observed in MDA-MB-435 cells, suggesting that the binding and internalization of LyP1NWs are mediated by p32 receptors on the surface of MDA-MB-435 cells. The lack of interaction of LyP1NWs with C8161 cells is presumed to be due to insufficient availability of p32 receptors on the cell surface (FIG. 14). As expected, control NWs exhibited no interaction in either cell type, regardless of the heat treatment.

The possibility of selective homing of LyP1NWs to heated xenograft tumors in vivo was then tested. Similar to the in vitro results, targeting of LyP1NWs to heated MDA-MB-435 tumors was prominent relative to unheated tumors, while the ability of LyP1NWs to home to heated C8161 tumors was not significantly different relative to the unheated tumors (FIG. 15). Histological analysis revealed large quantities of LyP1NWs occupying vessel structures that were not colocalized with the blood vessel stain, consistent with the previously reported affinity of LyP1 for lymphatics. In both types of tumors, most of the observed LyP1NWs were either colocalized with p32 receptors or distributed in the extravascular region of the heated tumors. Additionally, the distribution of control NWs in tumors did not correlate with the p32 receptor distribution, even though significant quantities of NWs were observed in the heated tumors. Furthermore, histological images of tumors for which LyP1NWs were administered immediately after heat treatment were similar to those for which LyP1NWs were injected right before heat treatment, suggesting that prominent targeting of LyP1NWs on the individual cells of heated tumors can be attributed mainly to their binding to the p32 receptors, not the simultaneous hyperthermia.

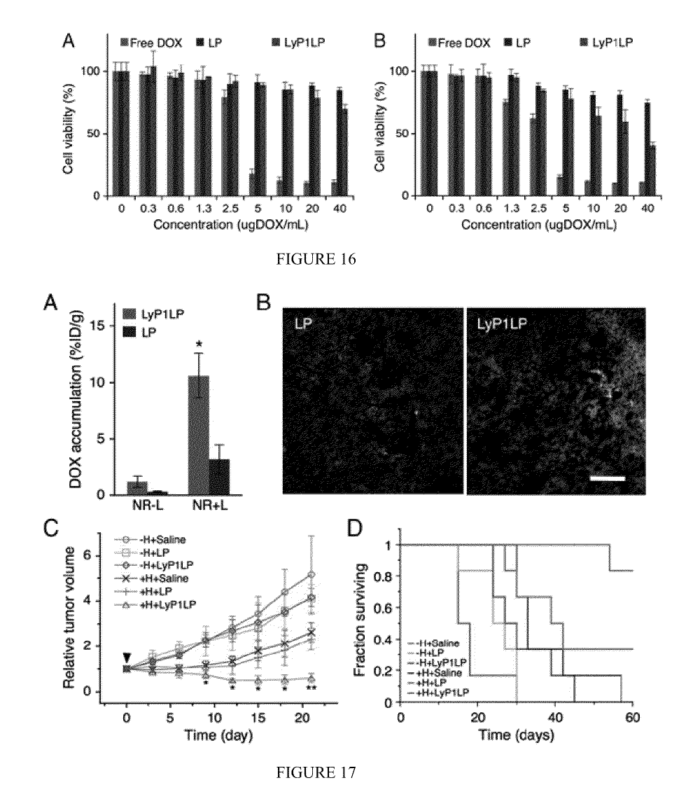

Having verified temperature-induced amplification of nanoparticle targeting to tumor cells in vitro and to xenografted tumors in vivo, in vitro photothermal-assisted cytotoxicity of targeted therapeutic carriers was evaluated. Liposomes constructed from lipids that are not thermally sensitive were prepared and loaded with the anti-cancer drug doxorubicin (DOX). The LyP1 peptide-conjugated DOX liposomes (LyP1LPs) displayed greater levels of cytotoxicity toward MDA-MB-435 cells relative to control DOX liposomes (DOX concentration >10 ug DOX/mL in both experiments). Enhanced cytotoxicity was observed for heat-treated (45° C.) samples, whereas the measured difference in cytotoxicity at 37° C. was insignificant (FIGS. 16a and 16b). The increased cytotoxicity of LyP1LPs toward heat-treated cells is ascribed to a combination of hyperthermal chemotherapy and targeting to (up regulated) receptor proteins. Although it was reported that LyP1 peptide itself has therapeutic effect, the peptide amount on the particles is much less than was needed for the anti-tumor activity. By contrast, the heat-induced cytotoxicity of LyP1LPs toward C8161 melanoma cells was significantly less pronounced; this is attributed to lower levels of expression of p32 on the C8161 cellular surface and higher resistance to DOX, relative to MDA-MB-435 cells.

The therapeutic efficacy of the complete cooperative nanomaterials system was tested on a xenograft mouse cancer model. Twenty-four hrs post-treatment, targeting efficacy of LyP1LPs was significantly larger in the photothermally engineered tumors than in the normal tumors and than that of control LPs (FIGS. 17a and b). The results show that targeted LPs display greater accumulation in the engineered tumors and deliver more encapsulated DOX payload relative to untargeted LPs. By contrast, in the normal (unheated) tumor environment, both LP formulations show relatively low levels of accumulation (FIG. 17a). Additionally, in order to achieve therapeutic effects in the unheated tumor, multiple administrations of relatively high doses of LPs are required. However, addition of the targeting ligand LyP-1 to the LP formulation slows tumor growth.

As mentioned above, hyperthermia in the temperature range ˜43° C. has been shown to selectively damage malignant cells relative to normal cells. Similarly, the increased temperature in the tumor produced by NR-mediated photothermal heating slows tumor growth in vivo, although it does not reduce tumor volume. However, tumors (or tumor cells) whose local microenvironment has been engineered by NR-mediated heating are more vulnerable to attack by therapeutic nanoparticles (FIGS. 17c and 17d). Combined with NR-mediated photothermal engineering, a single injection of therapeutic nanoparticles at a relatively low therapeutic dose (3 mg DOX/kg) is able to achieve significant tumor regression or elimination, which has not been observed in this tumor model with previous targeted therapies even with multiple high doses. For all the treatments studied above, no significant loss of body mass was observed.

The data demonstrates that the appropriate combination of nanomaterials currently under investigation in cancer therapy can significantly enhance therapeutic efficacy relative to the individual components. Site-specific photothermal heating of NRs can engineer the local tumor microenvironment to enhance the accumulation of therapeutic targeted liposomes, which increases the overall hyperthermal and chemotherapeutic tumor-destroying effects. This cooperative nanosystem holds clinical relevance because gold salts (for rheumatoid arthritis therapies) and doxorubicin-containing liposomes (Doxil®) have been approved for clinical use, and local hyperthemia is a well-established means of destroying diseased tissues in the human body. Although the liposomes in this study are similar to Doxil®, it should be pointed out that the gold nanorod and iron oxide nanoworm formulations used in the study are distinct from clinically approved gold or iron oxide materials. Accordingly cooperative, synergistic therapies using dual or multiple nanomaterials can significantly reduce the required dose of anti-cancer drugs, mitigating toxic side effects and more effectively eradiating drug-resistant cancers.

A number of embodiments of the disclosure have been described. Nevertheless, it will be understood that various modifications may be made without departing from the spirit and scope of the disclosure. Accordingly, other embodiments are within the scope of the following claims.

What is claimed is:

1. An elongated nanostructure comprising:
a plurality of nanostructures or nanoparticles conjugated or encapsulated to form an elongated structure having a first principle axis longer than the other two principle axes, with at least one dimension, such as length or diameter, between 1 and 200 nanometers.

2. The elongated nanostructure of claim 1, wherein the plurality of nanostructures or nanoparticles comprise a magnetic material.

3. The elongated nanostructure of claim 1, wherein the plurality of nanostructures or nanoparticles comprises an iron oxide.

4. The elongated nanostructure of claim 1, wherein the plurality of nanostructures are encapsulated in a biocompatible material.

5. The elongated nanostructure of claim 1, wherein the plurality of nanostructures or nanoparticles are conjugated to one another.

6. The elongated nanostructure of claim 4, wherein the biocompatible material is a dextran.

7. The elongated nanostructure of claim 1, further comprising a targeting moiety linked to the elongated nanostructure.

8. The elongated nanostructure of claim 7, wherein the targeting moiety is selected from the group consisting of a receptor ligand, an antibody, an antibody fragment, a small molecule and a peptide comprising 2 or more amino acids.

9. The elongated nanostructure of claim 8, wherein the peptide is a targeting moiety that interacts with a cognate on a cell comprising a cell proliferative disorder.

10. The elongated nanostructure of claim 8, wherein the targeting moiety is a peptide.

11. The elongated nanostructure of claim 9, wherein the targeting moiety is a peptide.

12. The elongated nanostructure of claim 10, wherein the targeting moiety comprises a sequence selected from the group consisting of SEQ ID NO:1 and SEQ ID NO:2.

13. A method of making an elongated nanostructure of claim 1, comprising precipitating a metal-containing or ceramic nanostructure or nanoparticle in a high molecular weight dextran.

14. The method of claim 13, wherein the method comprises precipitating iron oxide nanoparticles from a solution containing Fe²⁺_{(aq)}, Fe³⁺_{(aq)}, ammonia or other alkali solution, and a relatively low concentration of dextran.

15. The method of claim 14, wherein the dextran comprises a molecular weight of about 10-30 kDa.

16. An elongated nanostructure obtained by the method of claim 14.

17. The elongated nanostructure of claim 16, conjugated to a targeting moiety.

18. A method of imaging a cell, tissue or tumor comprising contacting a cell, tissue or tumor with an elongated nanostructure and imaging the cell, tissue or tumor.

19. A method of treating a tumor comprising contacting the tumor with an elongated nanostructure, causing the elongated nanostructures to heat at the site of the tumor, and contacting the tumor with a chemotherapeutic agent.

20. The method of claim 19, wherein the elongated nanostructure comprise a plurality of nanoparticles conjugated to one another.

21. The method of claim 20, wherein the plurality of nanoparticles are encapsulated in a biocompatible material.

22. The method of claim 21, wherein the biocompatible material is selected from the group consisting of a dextran, polyethylene glycol (PEG), polyvinyl pyrrolidone, and chitosan.

23. The method of claim 20, wherein elongated nanostructure further comprises a targeting moiety linked to the elongated nanostructure.

24. The method of claim 23, wherein the targeting moiety is selected from the group consisting of a receptor ligand, an antibody, an antibody fragment, a small molecule and a peptide comprising 2 or more amino acids.

25. The method of claim 24, wherein the peptide is a targeting moiety that interacts with a cognate on a cell comprising a cell proliferative disorder.

26. The method of claim 25, wherein the peptide comprises a sequence selected from the group consisting of SEQ ID NO:1, SEQ ID NO:2 or SEQ ID NO:3.

27. The method of claim 19, wherein the chemotherapeutic agent is delivered in a liposomal or micellar form.

28. The method of claim 27, wherein the chemotherapeutic agent is selected from the group consisting of doxorubicin, taxol and combretastatin.
